SEQUENCE LISTING (20 sequences)

SEQ ID NO: 1 - protein, 16 aa, synthetic construct
KSSQSLLDSDGKTYLN

SEQ ID NO: 2 - protein, 7 aa, synthetic construct
LVSKLDS

SEQ ID NO: 3 - protein, 9 aa, synthetic construct
WQGTYFPYT

SEQ ID NO: 4 - protein, 5 aa, synthetic construct
HTYAH

SEQ ID NO: 5 - protein, 17 aa, synthetic construct
RIDPANGNTKYDPKFQG

SEQ ID NO: 6 - protein, 10 aa, synthetic construct
DHYGSSLLDY

SEQ ID NO: 7 - protein, 112 aa, synthetic construct
DVVMTQTPLSLSVTPGQPASISCKSSQSLLDSDGKTYLNWFQQRPGQSPRRLIYLVSKLDSGVPDRFSGSGSGTDFTLKISRVEAEDVGVYYCWQGTYFPYTFGQGTKLEIK

SEQ ID NO: 8 - protein, 119 aa, synthetic construct
QVQLVQSGAEVKKPGASVKLSCKASGYNFKHTYAHWVRQAPGQGLEWIGRIDPANGNTKYDPKFQGRATMTADTASNTAYLELSSLRSEDTAVYYCVADHYGSSLLDYWGQGTLVTVSS

SEQ ID NO: 9 - protein, 219 aa, synthetic construct
DVVMTQTPLSLSVTPGQPASISCKSSQSLLDSDGKTYLNWFQQRPGQSPRRLIYLVSKLDSGVPDRFSGSGSGTDFTLKISRVEAEDVGVYYCWQGTYFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 446 aa, synthetic construct
QVQLVQSGAEVKKPGASVKLSCKASGYNFKHTYAHWVRQAPGQGLEWIGRIDPANGNTKYDPKFQGRATMTADTASNTAYLELSSLRSEDTAVYYCVADHYGSSLLDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 16 aa, synthetic construct
RSSQSIVHSNGNTYLE

SEQ ID NO: 12 - protein, 7 aa, synthetic construct
KVSNRFS

SEQ ID NO: 13 - protein, 9 aa, synthetic construct
FQGSHVPLT

SEQ ID NO: 14 - protein, 5 aa, synthetic construct
DYEMH

SEQ ID NO: 15 - protein, 17 aa, synthetic construct
VIESETGGTAYNQKFKG

SEQ ID NO: 16 - protein, 16 aa, synthetic construct
EGITTVATTYYWYFDV

SEQ ID NO: 17 - protein, 112 aa, synthetic construct [sequence omitted]

SEQ ID NO: 18 - protein, 125 aa, synthetic construct [sequence omitted]

SEQ ID NO: 19 - protein, 219 aa, synthetic construct
DVVMTQSPLSLPVTLGQPASISCRSSQSIVHSNGNTYLEWYLQKPGQSPQLLIYKVSNRFSGVPDRFSGSGSGTDFTLKISRVEAEDVGVYYCFQGSHVPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 452 aa, synthetic construct
QGQLVQSGAEVKKPGASVKVSCKASGYTFTDYEMHWVRQAPIHGLEWIGVIESETGGTAYNQKFKGRVTITADKSTSTAYMELSSLRSEDTAVYYCAREGITTVATTYYWYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

(19) United States
(12) Patent Application Publication
(10) Pub. No.: US 2023/0322929 A1
(43) Pub. Date: Oct. 12, 2023

(54) Compositions and Methods for Treating Solid Tumors with Anti-BTLA as Mono or Combination Therapy

(71) Applicant: SHANGHAI JUNSHI BIOSCIENCES CO., LTD., Shanghai (CN)

(72) Inventors: Sheng YAO, Columbia, MD (US); Hui FENG, Shanghai (CN)

(73) Assignee: SHANGHAI JUNSHI BIOSCIENCES CO., LTD., Shanghai (CN)

(21) Appl. No.: 18/297,983

(22) Filed: Apr. 10, 2023

Related U.S. Application Data
(60) Provisional application No. 63/330,089, filed on Apr. 12, 2022.

(51) Int. Cl. C07K 16/28; A61P 35/00

(52) U.S. Cl. CPC C07K 16/2818; A61P 35/00; C07K 16/2827; A61K 2039/54

(57) ABSTRACT

The present disclosure relates to the use of an anti-BTLA antagonist (including antibodies and antigen binding fragments) in the treatment of various tumors, either as a mono therapy or a combination therapy with other immune checkpoint inhibitor therapy such as anti-PD-1 or anti-PD-L1, and methods of selecting subjects in need of treatment. Preferably, various solid tumors are treated.

### CROSS-REFERENCE TO RELATED APPLICATION

This application claims the benefit and priority to U.S. Provisional Application No. 63/330,089, filed Apr. 12, 2022, the contents of which is incorporated by reference in its entirety herein.

### INCORPORATION BY REFERENCE

A sequence listing contained in the file named P35190US01.XML which is 20,224 bytes (measured in MS-Windows®) and created on Apr. 7, 2023, is filed electronically herewith and incorporated by reference in its entirety.

### BACKGROUND

BTLA (B and T lymphocyte attenuator) is a CD28 receptor family member identified in 2003 (Watanabe et al., Nat Immunol 4, 670-679 (2003)). It is expressed on T and B lymphocytes, and subsets of dendritic cells (DC).

Herpes virus entry mediator (HVEM), a TNF receptor widely expressed in hematopoietic system, was identified as a counter receptor for BTLA (Sedy et al., Nat Immunol 6, 90-98 (2005)). HVEM is expressed on T, B, NK, myeloid and dendric cells, and a variety of tumor cells including non-small cell lung cancer (NSCLC) (Ren et al., Lung Cancer 125, 115-120 (2018)), melanoma (Haymaker et al., Oncoimmunology 4, e1014246 (2015)), and lymphomas (Costello et al., Leukemia 17, 2500-2507 (2003); Pasero et al., Curr Opin Pharmacol 12, 478-485 (2012)). Tumor expression of HVEM has been associated with poor prognosis and immune escape (Inoue et al., Anticancer Res 35, 1361-1367 (2015); Ren et al., Lung Cancer 125, 115-120 (2018)).

Blockade of BTLA pathway by mAbs has been demonstrated in several syngeneic mouse models to enhance specific T cell responses and inhibit tumor growth. Study of peripheral blood mononuclear cells (PBMC) from melanoma patients and NSCLC patients revealed that BTLA is expressed at high levels on tumor specific cytotoxic T lymphocytes (CTLs) and inhibits T cell function upon engagement by tumor expressed HVEM (Derre et al., The Journal of clinical investigation 120, 157-167 (2010); Thommen et al., Cancer Immunol Res 3, 1344-1355 (2015)).

In terms of the currently existing immunotherapy, blockade of PD-1 and CTLA4 pathways by mAbs have demonstrated clinical efficacy against a broad spectrum of human malignancies. However, only a fraction of patients will benefit from such treatments and the improvement in clinical outcomes of approved PD-1/PD-L1 and CTLA-4 targeted therapy is still limited (Chowdhury et al., J Intern Med 283, 110-120 (2018)). Moreover, a large proportion of patients with initial response will eventually become refractory to treatment and the disease will progress.

Therefore, there is an unmet medical need for better alternative immunotherapy of anti-BTLA mAb that aims to promote T cell activation and thus anti-tumor response in treating human malignancies and solid tumors including lymphoma.

### SUMMARY

In an aspect, this application discloses a method for treating a solid tumor in a patient in need thereof, the method comprising: administering to the patient a pharmaceutically effective amount of an anti-BTLA antibody or antigen binding fragment thereof.

In an aspect, this application discloses a method of suppressing tumor growth in a patient in need thereof comprising administering to the patient an effective amount of an anti-BTLA antibody or antigen binding fragment thereof in combination with an effective amount of an anti-PD-1 or anti-PD-L1 antibody or antigen binding fragment thereof, wherein the anti-BTLA antibody or antigen binding fragment thereof and the anti-PD-1 or anti-PD-L1 antibody or antigen binding fragment thereof suppress the tumor growth in the patient.

In an aspect, this application discloses a method for treating a solid tumor in a patient in need thereof, the method comprising: administering to the patient a pharmaceutically effective amount of an inhibitor of the interaction between the BTLA receptor and one or more of its ligands.

### BRIEF DESCRIPTION OF THE DRAWINGS

FIG. 1 depicts an illustration of the schematic of Phase 1 study to evaluate TAB004 monotherapy, comprising Part A (dose escalation), and Part B (cohort expansion).

FIG. 2 depicts the mean serum concentration-time profiles following intravenous infusion of TAB004 at different doses (0.3, 1, 3, and 10 mg/kg) following a first dose (Cycle=C1), and after consecutive Q3W doses (Cycle=C4).

FIG. 3 depicts the timeline of treating an exemplary melanoma patient in the 3 mg/kg TAB004 dosing group, who showed partial response (PR) to the treatment and who is refractory to Nivolumab and Braf/Mek inhibitors.

FIG. 4 shows tumor suppression from TAB004 alone or combined with anti-PD-1 antibody (JS001, toripalimab) in a mouse MC38 xenograft model.

### DETAILED DESCRIPTION

Unless defined otherwise, all technical and scientific terms used herein have the same meaning as commonly understood by one of ordinary skill in the art to which this disclosure belongs.

All publications, patents, and patent applications mentioned in this specification are herein incorporated by reference to the same extent as if each individual publication, patent, or patent application was specifically and individually indicated to be incorporated by reference.

As used herein and in the appended claims, the singular forms “a,” “an,” and “the” include plural referents unless the context clearly dictates otherwise. By way of example, “an element” means at least one element and can include more than one element.

### General Definitions

As used herein, the term “substantially”, when used to modify a quality, generally allows certain degree of variation without that quality being lost. For example, in certain aspects such degree of variation can be less than 0.1%, about 0.1%, about 0.2%, about 0.3%, about 0.4%, about 0.5%, about 0.6%, about 0.7%, about 0.8%, about 0.9%, about 1%, between 1-2%, between 2-3%, between 3-4%, between 4-5%, or greater than 5% or 10%.

The term “about”, when modifying the quantity (e.g., mg) of a substance or composition, or the value of a parameter characterizing a step in a method, or the like, refers to variation in the numerical quantity that can occur, for example, through typical measuring, handling and sampling procedures involved in the preparation, characterization and/or use of the substance or composition; through inadvertent error in these procedures; through differences in the manufacture, source, or purity of the ingredients employed to make or use the compositions or carry out the procedures; and the like. In certain aspects, “about” can mean a variation of ±0.1%, ±0.5%, ±1%, ±2%, ±3%, ±4%, ±5%, ±6%, ±7%, ±8%, ±9% or ±10%. When referring to the dosage of “about 200 mg,” the dosage can be from 180 mg to 220 mg, from 190 mg to 210 mg, from 195 mg to 205 mg, from 200 mg to 220 mg, or from 190 mg to 220 mg. It alternative aspects, the dosage can be 180 mg, 185 mg, 190 mg, 195 mg, 200 mg, 205 mg, 210 mg, 215 mg, or 220 mg. When referring to the amount of time between administrations in a therapeutic treatment regimen (e.g., amount of time between administrations of an anti-BTLA antibody or antigen binding fragment thereof, e.g. “about 3 weeks,” which is used interchangeably herein with “approximately every three weeks”), “about” refers to the stated time±a variation that can occur due to patient/clinician scheduling and availability around the 3-week target date. For example, “about 3 weeks” can refer to 3 weeks±4 days, 3 weeks±3 days, 3 weeks±2 days or 3 weeks±1 day, or may refer to 2 weeks, 3 days through 3 weeks, 4 days.

Where a range of values is provided, it is understood that each intervening value, between the upper and lower limit of that range and any other stated or intervening value in that stated range is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either both of those included limits are also included in the disclosure.

As used herein, an “anti-BTLA therapy” can mean either an anti-BTLA mono-therapy or an anti-BTLA combo-therapy. An “anti-BTLA mono-therapy” refers to the use of an BTLA antagonist (e.g., an anti-BTLA antibody) alone without another active pharmaceutical or biological ingredient for treating a disease or condition in a patient. An “anti-BTLA combo-therapy” refers to the combined use of an BTLA antagonist (e.g., an anti-BTLA antibody) and another active pharmaceutical or biological ingredient (e.g., a chemotherapeutic or another immune checkpoint inhibitor such as an anti-PD-1 antibody) for treating a disease or condition in a patient.

As used herein, an “adjuvant therapy” refers to additional cancer treatment given after the primary treatment to lower the risk that the cancer will come back. Adjuvant therapy may include chemotherapy, radiation therapy, hormone therapy, targeted therapy, or biological therapy.

The term “patient” (alternatively referred to as “subject” or “individual” herein) refers to a mammal (e.g., rat, mouse, dog, cat, rabbit) capable of being treated with the methods and compositions of the disclosure, most preferably a human. In an aspect, the patient is an adult patient. In other aspects, the patient is a pediatric patient.

The terms “cancer”, “tumor”, “cancerous”, or “malignant” refer to or describe the physiological condition in mammals that is typically characterized by unregulated cell growth. Examples of cancer include but are not limited to, carcinoma, lymphoma, leukemia, blastoma, and sarcoma. More particular examples of such cancers include, but are not limited to, squamous cell carcinoma, myeloma, small-cell lung cancer, non-small cell lung cancer, glioma, Hodgkin lymphoma, non-hodgkin's lymphoma, acute myeloid leukemia (AML), multiple myeloma, gastrointestinal (tract) cancer, renal cancer, ovarian cancer, liver cancer, lymphoblastic leukemia, lymphocytic leukemia, colorectal cancer, endometrial cancer, kidney cancer, prostate cancer, thyroid cancer, melanoma, chondrosarcoma, neuroblastoma, pancreatic cancer, glioblastoma multiforme, cervical cancer, brain cancer, stomach cancer, bladder cancer, hepatoma, breast cancer, colon carcinoma, and head and neck cancer. Additional cancers that may be treated in accordance with the present disclosure include those characterized by elevated expression of one or more of BTLA, HVEM, PD-L1 and PD-L2 in tested tissue samples.

As used herein, the term “treating” refers to (i) completely or partially inhibiting a disease, disorder or condition, for example, arresting its development; (ii) completely or partially relieving a disease, disorder or condition, for example, causing regression of the disease, disorder and/or condition; or (iii) completely or partially preventing a disease, disorder or condition from occurring in a patient that may be predisposed to the disease, disorder and/or condition, but has not yet been diagnosed as having it. Similarly, “treatment” refers to both therapeutic treatment and prophylactic or preventative measures.

As used herein, “treat” or “treating” a cancer or tumor means to administer an antibody or antigen binding fragment, without or without another agent (e.g., another antibody or antigen binding fragment recognizing another target, or a chemotherapy agent) to a subject having a cancer, or diagnosed with a cancer, to achieve at least one positive therapeutic effect, such as for example, reduced number of cancer cells, reduced tumor size, reduced rate of cancer cell infiltration into peripheral organs, or reduced rate of tumor metastasis or tumor growth. “Treatment” of a cancer or tumor may include one or more of the following: inducing/increasing an antitumor immune response, decreasing the number of one or more tumor markers, halting or delaying the growth of a tumor or blood cancer or progression of cancer, stabilization of a cancer disease condition, inhibiting the growth or survival of tumor cells, eliminating or reducing the size of one or more cancerous lesions or tumors, decreasing the level of one or more tumor markers, ameliorating or abrogating the clinical manifestations of cancer, reducing the severity or duration of the clinical symptoms of cancer, prolonging the survival of a patient relative to the expected survival in a similar untreated patient, and inducing complete or partial remission of a cancerous condition.

As used herein, an “antibody” refers to any form of antibody, either full-length or fragment(s) thereof, that exhibits the desired biological or binding activity. Thus, it is used in the broadest sense and specifically covers, but is not limited to, monoclonal antibodies (including full length monoclonal antibodies), polyclonal antibodies, humanized, fully human antibodies, chimeric antibodies, fully synthetic antibodies, and single chain antibodies. Non-human antibodies can be humanized by recombinant methods to reduce their immunogenicity in humans.

In general, the basic antibody structural unit comprises a tetramer. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. The variable regions of each light/heavy chain pair form the antibody binding site. Thus, in general, an intact antibody has two binding sites. Except in bifunctional or bispecific antibodies, the two binding sites are, in general, the same.

Typically, the variable domains of both the heavy and light chains comprise three hypervariable regions, also called complementarity determining regions (CDRs), which are located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3 and FR4. The assignment of amino acids to each domain is, generally, in accordance with the definitions of Sequences of Proteins of Immunological Interest. See, e.g., Kabat Adv. Prot. Chem. 32: 1-75 (1978); Kabat et al., J Biol. Chem. 252:6609-6616 (1977); Chothia et al., J. Mol. Biol. 196:901-917 (1987) or Chothia et al., Nature 342:878-883 (1989).

The term “hypervariable region” refers to the amino acid residues of an antibody that are responsible for antigen-binding. The hypervariable region comprises amino acid residues from a “complementarity determining region” or “CDR” (i.e. CDRL1, CDRL2 and CDRL3 in the light chain variable domain and CDRH1, CDRH2 and CDRH3 in the heavy chain variable domain). See Kabat et al. (1991) Sequences of Proteins of Immunological Interest, 5th Ed. Public Health Service, National Institutes of Health, Bethesda, Md. (defining the CDR regions of an antibody by sequence); see also Chothia and Lesk, J. Mol. Biol. 196: 901-917 (1987) (defining the CDR regions of an antibody by structure). The term “framework” or “FR” residues refers to those variable domain residues other than the hypervariable region residues defined herein as CDR residues.

As used herein, an “antibody fragment” or “antigen binding fragment” refers to antigen binding fragments of antibodies, i.e. antibody fragments that retain the ability to specifically bind to the antigen bound by the full-length antibody, e.g. fragments that retain one or more CDR regions. Examples of antibody binding fragments include, but are not limited to, Fab, Fab′, F(ab′)2, and Fv fragments, diabodies, linear antibodies, single-chain antibody molecules, nanobodies and multispecific antibodies formed from antibody fragments.

An antibody that “specifically binds to” a specified target protein is an antibody that exhibits preferential binding to that target as compared to other proteins, but this specificity does not require absolute binding specificity. An antibody is considered “specific” for its intended target if its binding is determinative of the presence of the target protein in a sample, e.g. without producing undesired results such as false positives. Antibodies, or binding fragments thereof, useful in the present disclosure will bind to the target protein with an affinity that is at least two fold greater, preferably at least ten times greater, more preferably at least 20-times greater, and most preferably at least 100-times greater than the affinity with non-target proteins.

“Chimeric antibody” refers to an antibody in which a portion of the heavy and/or light chain is identical with or homologous to corresponding sequences in an antibody derived from a particular species (e.g., human) or belonging to a particular antibody class or subclass, while the remainder of the chain(s) is identical with or homologous to corresponding sequences in an antibody derived from another species (e.g., mouse) or belonging to another antibody class or subclass, as well as fragments of such antibodies, so long as they exhibit the desired biological activity.

“Human antibody” refers to an antibody that comprises human immunoglobulin protein sequences only. A human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences, respectively.

“Humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum”, “hu” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions may be included to increase affinity, increase stability of the humanized antibody, or for other reasons.

“Isolated antibody” and “isolated antibody fragment” refers to the purification status and in such context means the named molecule is substantially free of other biological molecules such as nucleic acids, proteins, lipids, carbohydrates, or other material such as cellular debris and growth media. Generally, the term “isolated” is not intended to refer to a complete absence of such material or to an absence of water, buffers, or salts, unless they are present in amounts that substantially interfere with experimental or therapeutic use of the binding compound as described herein.

“CDR” or “CDRs” means complementarity determining region(s) in an immunoglobulin variable region, generally defined using the Kabat numbering system. “Kabat,” as used herein, means an immunoglobulin alignment and numbering system pioneered by Elvin A. Kabat (Sequences of Proteins of Immunological Interest, 5th Ed. Public Health Service, National Institutes of Health, Bethesda, Md. (1991)).

“Monoclonal antibody” or “mAb” or “Mab”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method first described by Kohler et al., Nature 256: 495 (1975), or may be made by recombinant DNA methods (see, e.g., ET.S. Pat. No. 4,816,567). The “monoclonal antibodies” may also be isolated from phage antibody libraries using the techniques described in Clackson et al., Nature 352: 624-628 (1991) and Marks et al., J Mol. Biol. 222: 581-597 (1991), for example. See also Presta J Allergy Clin. Immunol. 116:731 (2005).

“Conservatively modified variants” or “conservative substitution” refers to substitutions of amino acids in a protein with other amino acids having similar characteristics (e.g. charge, side-chain size, hydrophobicity/hydrophilicity, backbone conformation and rigidity, etc.), such that the changes can frequently be made without altering the biological activity or other desired property of the protein, such as antigen affinity and/or specificity. Those of skill in this art recognize that, in general, single amino acid substitutions in non-essential regions of a polypeptide do not substantially alter biological activity (see, e.g., Watson et al., Molecular Biology of the Gene, The Benjamin/Cummings Pub. Co., p. 224 (4th Ed.) (1987)). In addition, substitutions of structurally or functionally similar amino acids are less likely to disrupt biological activity.

The term “intravenous infusion” refers to introduction of an agent into the vein of an animal or human patient over a period of time greater than approximately 15 minutes, generally between approximately 30 to 90 minutes. Generally, this is achieved via an intravenous (IV) bag.

### BTLA

Herein, the terms “B and T lymphocyte attenuator” and “BTLA” gene/protein can be used interchangeably and include variants, isotypes, homologues, orthologs, and paralogs. For example, in an aspect, a human BTLA-specific antibody can cross-react with BTLA from a non-human species. In other aspects, a human BTLA-specific antibody may be fully specific for human BTLA and have no species cross-reactivity or other types of cross-reactivities. Unless otherwise stated, the term “human BTLA” or “hBTLA” refers to the human BTLA sequence. Unless otherwise stated, the human BTLA sequence includes all human isotypes and BTLA variants, e.g., the complete amino acid sequence of human BTLA with Genbank accession No. AAP44003. There are also at least two human BTLA transcript variants, transcript variant 1 and transcript variant 2. The former encodes a protein that is 289 amino acids in length (GenBank accession No. NP_861445) and has nearly 98% identity to the BTLA sequence with an accession No. AAP44003, and the latter encodes a protein that is 241 amino acids in length (GenBank accession No. NP_001078826).

BTLA is a negative regulator of immune response with a C-terminal inhibitory motif involved in the inhibition of IL-2 production and T cell expansion (Watanabe et al., Nat. Immunol., 4, 670-679, (2003); Chemnitz et al., J. Immunol., 176, 6603-6614, (2006)). In an aspect, an anti-BTLA antibody disclosed or used here in recognizes an epitope in the extracellular domain of BTLA.

As used herein, the term “anti-BTLA antibody” or “anti-BTLA antigen binding fragment” refers to an antibody that binds to BTLA and partially, substantially or completely blocks the binding of BTLA to HVEM, including fragments or derivatives of the antibody, typically including at least one fragment of an antigen-binding region or an variable region (e.g., one or more CDRs) of the antibody, which retains at least some binding specificities of the antibody. Examples of antigen binding fragments include, but are not limited to, Fab, Fab′, F(ab′)₂, and Fv fragments; diabodies; linear antibodies; single chain antibody molecules, such as sc-Fv; nanobodies and multispecific antibodies formed from antibody fragments. A binding fragment or a derivative typically retains at least 10% of its BTLA binding activity when the BTLA binding activity is expressed on a molar concentration basis. Preferably, a binding fragment or a derivative retains at least 20%, 50%, 70%, 80%, 90%, 95%, or 100% or more of the BTLA binding affinity of the antibody. It is also contemplated that anti-BTLA antigen-binding fragments may include conservative or non-conservative amino acid substitutions (referred to as “conservative variants” or “function-conservative variants” of antibodies) that do not significantly alter their biological activity.

### BTLA Antagonists

In an aspect, an anti-BTLA therapy disclosed herein uses an anti-BTLA antibody (or an antigen-binding fragment thereof) comprising one or more properties selected from the following: A) completely blocking the binding of BTLA to HVEM; B) cross-reacting with cynomolgus monkey BTLA; C) binding to human BTLA with a K_{D}≤0.28 nM; and D) having no ability to mediate Antibody-Dependent Cellular Cytotoxicity (ADCC) effect. In another aspect, an anti-BTLA antibody or antigen-binding fragment thereof from US20210246209A1 is used in an anti-BTLA therapy.

In an aspect, an anti-BTLA therapy disclosed herein uses an anti-BTLA antibody (or an antigen-binding fragment thereof) comprising a light chain variable region comprising LCDR1, LCDR2 and LCDR3 as shown in SEQ ID NO:1, SEQ ID NO:2 and SEQ ID NO: 3, respectively; and comprising a heavy chain variable region comprising HCDR1, HCDR2 and HCDR3 as shown in SEQ ID NO:4, SEQ ID NO:5, and SEQ ID NO:6, respectively. In another aspect, one of more of the foregoing CDRs comprises one or more, two or more, or three or more conservative substitutions relative to SEQ ID Nos: 1 to 6. In another aspect, one of more of the foregoing CDRs comprises one or more, two or more, or three or more substitutions relative to SEQ ID NOs: 1 to 6.

In an aspect, an anti-BTLA therapy disclosed herein uses an anti-BTLA antibody (or an antigen-binding fragment thereof) comprising a light chain variable region sequence having at least 80%, 85%, 90%, 91%, 92%, 93%, 94%, 95%, 96%, 97%, 98%, 99% identity to SEQ ID NO:7 and a heavy chain variable region sequence having at least 80%, 85%, 90%, 91%, 92%, 93%, 94%, 95%, 96%, 97%, 98%, 99% identity to SEQ ID NO:8.

In an aspect, an anti-BTLA therapy disclosed herein uses an anti-BTLA antibody (or an antigen-binding fragment thereof) comprising a light chain variable region sequence of SEQ ID NO:7 and a heavy chain variable region sequence of SEQ ID NO:8. In another aspect, an anti-BTLA antibody or antigen-binding fragment thereof comprises at least 1, 2, 3, 4, 5, 6, 7, 8, 9, 10, 12, 14, 16, 18, 20, 22, 25, or 30 conservative substitutions relative to SEQ ID Nos: 7 and 8. In another aspect, an anti-BTLA antibody or antigen-binding fragment thereof comprises at least 1, 2, 3, 4, 5, 6, 7, 8, 9, 10, 12, 14, 16, 18, 20, 22, 25, or 30 substitutions relative to SEQ ID Nos: 7 and 8. In another aspect, an anti-BTLA antibody or antigen-binding fragment thereof comprises at most 1, 2, 3, 4, 5, 6, 7, 8, 9, 10, 12, 14, 16, 18, 20, 22, 25, or 30 conservative substitutions relative to SEQ ID Nos: 7 and 8. In another aspect, an anti-BTLA antibody or antigen-binding fragment thereof comprises at most 1, 2, 3, 4, 5, 6, 7, 8, 9, 10, 12, 14, 16, 18, 20, 22, 25, or 30 substitutions relative to SEQ ID Nos: 7 and 8.

In an aspect, an anti-BTLA therapy disclosed herein uses an anti-BTLA antibody (or its antigen-binding fragment thereof) which comprises a light chain sequence having at least 80%, 85%, 90%, 91%, 92%, 93%, 94%, 95%, 96%, 97%, 98%, 99% identity to SEQ ID NO:9 and a heavy chain sequence having at least 80%, 85%, 90%, 91%, 92%, 93%, 94%, 95%, 96%, 97%, 98%, 99% identity to SEQ ID NO:10.

In an aspect, an anti-BTLA therapy disclosed herein uses an anti-BTLA antibody such as TAB004 (also named or referenced as icatolimab, “JS 004”, “JS-004”, “JS004”, “TAB 004”, “TAB-004” or tifcemalimab; or its antigen-binding fragment thereof) which comprises a light chain sequence of SEQ ID NO:9 and a heavy chain sequence of SEQ ID NO:10. In another aspect, an anti-BTLA antibody or antigen-binding fragment thereof comprises at least 1, 2, 3, 4, 5, 6, 7, 8, 9, 10, 12, 14, 16, 18, 20, 22, 25, or 30 conservative substitutions relative to SEQ ID Nos: 9 and 10. In another aspect, an anti-BTLA antibody or antigen-binding fragment thereof comprises at least 1, 2, 3, 4, 5, 6, 7, 8, 9, 10, 12, 14, 16, 18, 20, 22, 25, or 30 substitutions relative to SEQ ID Nos: 9 and 10. In another aspect, an anti-BTLA antibody or antigen-binding fragment thereof comprises at most 1, 2, 3, 4, 5, 6, 7, 8, 9, 10, 12, 14, 16, 18, 20, 22, 25, or 30 conservative substitutions relative to SEQ ID Nos: 9 and 10. In another aspect, an anti-BTLA antibody or antigen-binding fragment thereof comprises at most 1, 2, 3, 4, 5, 6, 7, 8, 9, 10, 12, 14, 16, 18, 20, 22, 25, or 30 substitutions relative to SEQ ID Nos: 9 and 10.

Certain sequences exemplified above are shown in the following Table 1.

TABLE 1
CDRs, VH, VL, and heavy chain (HC) and light chain (LC)
sequences of anti-BTLA TAB004. The listed CDRs are based
on the Kabat numbering system.

| Name | Sequence | SEQ ID NO |
|---|---|---|
| LCDR1 | KSSQSLLDSDGKTYLN | SEQ ID NO: 1 |
| LCDR2 | LVSKLDS | SEQ ID NO: 2 |
| LCDR3 | WQGTYFPYT | SEQ ID NO: 3 |
| HCDR1 | HTYAH | SEQ ID NO: 4 |
| HCDR2 | RIDPANGNTKYDPKFQG | SEQ ID NO: 5 |
| HCDR3 | DHYGSSLLDY | SEQ ID NO: 6 |
| VL | DVVMTQTPLSLSVTPGQPASISCKSSQSLLDSDGK | SEQ ID NO: 7 |
| | TYLNWFQQRPGQSPRRLIYLVSKLDSGVPDRFSG | |
| | SGSGTDFTLKISRVEAEDVGVYYCWQGTYFPYTF | |
| | GQGTKLEIK | |
| VH | QVQLVQSGAEVKKPGASVKLSCKASGYNFKHTY | SEQ ID NO: 8 |
| | AHWVRQAPGQGLEWIGRIDPANGNTKYDPKFQG | |
| | RATMTADTASNTAYLELSSLRSEDTAVYYCVAD | |
| | HYGSSLLDYWGQGTLVTVSS | |
| LC | DVVMTQTPLSLSVTPGQPASISCKSSQSLLDSDGK | SEQ ID NO: 9 |
| | TYLNWFQQRPGQSPRRLIYLVSKLDSGVPDRFSG | |
| | SGSGTDFTLKISRVEAEDVGVYYCWQGTYFPYTF | |
| | GQGTKLEIKRTVAAPSVFIFPPSDEQLKSGTASVV | |
| | CLLNNFYPREAKVQWKVDNALQSGNSQESVTEQ | |
| | DSKDSTYSLSSTLTLSKADYEKHKVYACEVTHQ | |
| | GLSSPVTKSFNRGEC | |
| HC | QVQLVQSGAEVKKPGASVKLSCKASGYNFKHTY | SEQ ID NO: 10 |
| | AHWVRQAPGQGLEWIGRIDPANGNTKYDPKFQG | |
| | RATMTADTASNTAYLELSSLRSEDTAVYYCVAD | |
| | HYGSSLLDYWGQGTLVTVSSASTKGPSVFPLAPC | |
| | SRSTSESTAALGCLVKDYFPEPVTVSWNSGALTS | |
| | GVHTFPAVLQSSGLYSLSSVVTVPSSSLGTKTYTC | |
| | NVDHKPSNTKVDKRVESKYGPPCPPCPAPEFLGG | |
| | PSVFLFPPKPKDTLMISRTPEVTCVVVDVSQEDPE | |
| | VQFNWYVDGVEVHNAKTKPREEQFNSTYRVVS | |
| | VLTVLHQDWLNGKEYKCKVSNKGLPSSIEKTISK | |
| | AKGQPREPQVYTLPPSQEEMTKNQVSLTCLVKGF | |
| | YPSDIAVEWESNGQPENNYKTTPPVLDSDGSFFL | |
| | YSRLTVDKSRWQEGNVFSCSVMHEALHNHYTQ | |
| | KSLSLSLGK | |

In an aspect, an anti-BTLA therapy comprises the use of an anti-BTLA antibody or antigen-binding fragment thereof comprising a Fab fragment identical or substantially identical to the Fab fragment of icatolimab. A “Fab fragment” consists of a light chain, a CH1 and a variable region of a heavy chain. The heavy chain of a Fab molecule cannot form a disulfide bond with another heavy chain molecule.

In an aspect, an anti-BTLA therapy comprises the use of icatolimab with one or more, two or more, three or more, or four or more amino acid substitutions in the Fc region. In some aspect, such amino acid substitution(s) affect post-translational modification, e.g., glycosylation. An “Fc” region contains 2 heavy chain fragments comprising the CH1 and CH2 domains of an antibody. The 2 heavy chain fragments are held together by two or more disulfide bonds and by the hydrophobic interaction of the CH3 domains.

In an aspect, an anti-BTLA therapy comprises the use of a molecule comprising an Fab′ fragment of icatolimab or an Fab′ fragment substantially identical thereto. An “Fab′ fragment” contains a light chain and a heavy chain portion or fragment comprising the VH domain, the CH1 domain and the region between the CH1 and CH2 domains, such that interchain disulfide bonds can be formed between 2 heavy chains of 2 Fab' fragments to form an F(ab′)₂ molecule.

In an aspect, an anti-BTLA therapy comprises the use of a molecule comprising an F(ab′)₂ fragment of icatolimab or an F(ab′)₂ fragment substantially identical thereto. An “F(ab′)₂ fragment” contains 2 light chains and 2 heavy chains containing a portion of the constant region between the CH1 and CH2 domains, such that interchain disulfide bonds are formed between the 2 heavy chains. Thus, an F(ab′)₂ fragment consists of 2 Fab′ fragments held together by disulfide bonds between 2 heavy chains.

In an aspect, an anti-BTLA therapy comprises the use of a molecule comprising the Fv region of icatolimab. An “Fv region” comprises variable regions derived from both the heavy and light chains, but lacks constant regions.

In an aspect, an anti-BTLA therapy comprises the use of a scFv derived from icatolimab. As used herein, the term “single chain Fv” or “scFv” antibody refers to an antibody fragment comprising the VH and VL domains of the antibody, wherein these domains are present in a single polypeptide chain. An scFv polypeptide also typically comprise a polypeptide linker between the VH and the VL domains that enables the scFv to form the desired structure for antigen-binding.

In an aspect, an anti-BTLA therapy comprises the use of a domain antibody derived from icatolimab. A “domain antibody” is an immunofunctional immunoglobulin fragment that contains only the variable region of the heavy chain or the light chain. In some cases, two or more VH regions are covalently linked to a peptide linker to form a bivalent domain antibody. The 2 VH regions of the bivalent domain antibody may target the same or different antigens.

In an aspect, an anti-BTLA therapy comprises the use of a bivalent antibody derived from icatolimab. A “bivalent antibody” comprises 2 antigen-binding sites. In some cases, the 2 binding sites have the same antigen specificity. However, a bivalent antibody may be bispecific.

In an aspect, an anti-BTLA therapy comprises the use of a diabody derived from icatolimab. As used herein, the term “diabody” refers to a small antibody fragment having two antigen-binding sites, which comprises a heavy chain variable domain (VH) linked to a light chain variable domain (VL) in one polypeptide chain (VH-VL or VL-VH). By using a linker that is too short for pairing between two domains in one chain, the domains are forced to pair with the complementary domains of the other chain to form two antigen-binding sites.

In another aspect, other anti-BTLA antibodies (or their antigen-binding fragments) that do not necessarily block (or at least not completely block) BTLA and HVEM binding can also be used as disclosed herein for TAB004. Such additional anti-BTLA antibodies include ANB032 and LY3361237. In another aspect, an anti-BTLA antibody is HFB200603. In an aspect, an additional anti-BTLA antibody cross-competes with TAB004 for BTLA binding or the blocking effect over the BTLA/HVEM binding. In another aspect, an additional anti-BTLA antibody has substantially similar or identical affinity to BTLA as TAB004. In a further aspect, an additional anti-BTLA antibody has a higher affinity to BTLA compared to TAB004. In an aspect, an additional anti-BTLA antibody neutralizes the interaction between BTLA and HVEM to a similar extent as TAB004. In another aspect, an additional anti-BTLA antibody recognizes a BTLA epitope sequence or site(s) that overlaps with the TAB004's BTLA epitope sequence or site(s). In an aspect, an additional anti-BTLA antibody neutralizes or inhibits the interaction between BTLA and TAB004.

### PD-1/PD-L1

In an aspect, an anti-BTLA combo-therapy comprises the use of both an anti-BTLA antibody and an anti-PD-1 antibody, or a bispecific antibody targeting both BTLA and PD-1. An “anti-PD-1 antibody” useful in the present disclosure include monoclonal antibodies (mAb), or antigen binding fragments thereof, which specifically bind to human PD-1. Alternative names or synonyms for PD-1 and its ligands include: PDCD1, PD1, CD279 and SLEB2 for PD-1; PDCD1L1, PDL1, B7H1, B7-4, CD274 and B7-H for PD-L1; and PDCD1L2, PDL2, B7-DC, Btdc and CD273 for PD-L2.

In any of the treatment methods, compositions and uses of the present disclosure in which a human individual is being treated, the PD-1 antibody or antigen binding fragment thereof is a PD-1 antagonist that blocks binding of human PD-L1 to human PD-1, or blocks binding of both human PD-L1 and PD-L2 to human PD-1. Human PD-1 amino acid sequences can be found in NCBI Locus No. : NP 005009. Human PD-L1 and PD-L2 amino acid sequences can be found in NCBI Locus No.: NP_054862 and NP_079515, respectively. An anti-PD-1 antibody may be a human antibody, a humanized antibody or a chimeric antibody, and may include a human constant region. In an aspect the human constant region is selected from the group consisting of IgG1, IgG2, IgG3 and IgG4 constant regions, and in preferred aspects, the human constant region is an IgG1 or IgG4 constant region. In an aspect, the antigen binding fragment is selected from the group consisting of Fab, Fab′, F(ab′)₂, scFv and Fv fragments.

In an aspect, an anti-BTLA combo-therapy disclosed herein comprises an anti-PD-1 antibody such as nivolumab, pembrolizumab, toripalimab, sintilimab, camrelizumab, tislelizumab, or cemiplimab, or (i) a variant or antigen binding fragment or (ii) a biosimilar molecule of any of the foregoing. In another aspect, an anti-BTLA combo-therapy disclosed herein comprises an anti-PD-L1 antibody such as atezolizumab, durvalumab, or avelumab or (i) a variant or antigen binding fragment or (ii) a biosimilar molecule of any of the foregoing.

In an aspect, an anti-BTLA combo-therapy disclosed herein uses an anti-PD-1 antibody (or an antigen-binding fragment thereof) comprising a light chain variable region comprising LCDR1, LCDR2 and LCDR3 as shown in SEQ ID NO:11, SEQ ID NO:12 and SEQ ID NO: 13, respectively; and comprising a heavy chain variable region comprising HCDR1, HCDR2 and HCDR3 as shown in SEQ ID NO:14, SEQ ID NO:15, and SEQ ID NO:16, respectively. In another aspect, one of more of the foregoing CDRs comprises one or more, two or more, or three or more conservative substitutions relative to SEQ ID Nos: 11 to 16. In another aspect, one of more of the foregoing CDRs comprises one or more, two or more, or three or more substitutions relative to SEQ ID Nos: 11 to 16.

In an aspect, an anti-BTLA combo-therapy disclosed herein uses an anti-PD-1 antibody (or an antigen-binding fragment thereof) comprising a light chain variable region sequence having at least 80%, 85%, 90%, 91%, 92%, 93%, 94%, 95%, 96%, 97%, 98%, 99% identity to SEQ ID NO:17 and a heavy chain variable region sequence having at least 80%, 85%, 90%, 91%, 92%, 93%, 94%, 95%, 96%, 97%, 98%, 99% identity to SEQ ID NO:18.

In an aspect, an anti-BTLA combo-therapy disclosed herein uses an anti-PD-1 antibody (or an antigen-binding fragment thereof) comprising a light chain variable region sequence of SEQ ID NO:17 and a heavy chain variable region sequence of SEQ ID NO:18. In another aspect, an anti-PD-1 antibody or antigen-binding fragment thereof comprises at least 1, 2, 3, 4, 5, 6, 7, 8, 9, 10, 12, 14, 16, 18, 20, 22, 25, or 30 conservative substitutions relative to SEQ ID Nos: 17 and 18. In another aspect, an anti-PD-1 antibody or antigen-binding fragment thereof comprises at least 1, 2, 3, 4, 5, 6, 7, 8, 9, 10, 12, 14, 16, 18, 20, 22, 25, or 30 substitutions relative to SEQ ID Nos: 17 and 18. In another aspect, an anti-PD-1 antibody or antigen-binding fragment thereof comprises at most 1, 2, 3, 4, 5, 6, 7, 8, 9, 10, 12, 14, 16, 18, 20, 22, 25, or 30 conservative substitutions relative to SEQ ID Nos: 17 and 18. In another aspect, an anti-PD-1 antibody or antigen-binding fragment thereof comprises at most 1, 2, 3, 4, 5, 6, 7, 8, 9, 10, 12, 14, 16, 18, 20, 22, 25, or 30 substitutions relative to SEQ ID Nos: 17 and 18.

In an aspect, an anti-BTLA combo-therapy disclosed herein uses an anti-PD-1 antibody (or its antigen-binding fragment thereof) which comprises a light chain sequence having at least 80%, 85%, 90%, 91%, 92%, 93%, 94%, 95%, 96%, 97%, 98%, 99% identity to SEQ ID NO:19 and a heavy chain sequence having at least 80%, 85%, 90%, 91%, 92%, 93%, 94%, 95%, 96%, 97%, 98%, 99% identity to SEQ ID NO:20.

In an aspect, an anti-BTLA combo-therapy disclosed herein uses an anti-PD-1 antibody such as toripalimab (or its antigen-binding fragment thereof) which comprises a light chain sequence of SEQ ID NO:19 and a heavy chain sequence of SEQ ID NO:20. In another aspect, an anti-PD-1 antibody or antigen-binding fragment thereof comprises at least 1, 2, 3, 4, 5, 6, 7, 8, 9, 10, 12, 14, 16, 18, 20, 22, 25, or 30 conservative substitutions relative to SEQ ID Nos: 19 and 20. In another aspect, an anti-PD-1 antibody or antigen-binding fragment thereof comprises at least 1, 2, 3, 4, 5, 6, 7, 8, 9, 10, 12, 14, 16, 18, 20, 22, 25, or 30 substitutions relative to SEQ ID Nos: 19 and 20. In another aspect, an anti-PD-1 antibody or antigen-binding fragment thereof comprises at most 1, 2, 3, 4, 5, 6, 7, 8, 9, 10, 12, 14, 16, 18, 20, 22, 25, or 30 conservative substitutions relative to SEQ ID Nos: 19 and 20. In another aspect, an anti-PD-1 antibody or antigen-binding fragment thereof comprises at most 1, 2, 3, 4, 5, 6, 7, 8, 9, 10, 12, 14, 16, 18, 20, 22, 25, or 30 substitutions relative to SEQ ID Nos: 19 and 20.

Certain sequences exemplified above are shown in the following Table 2.

TABLE 2
CDRs, VH, VL, and heavy chain (HC) and light chain (LC)
sequences of anti-PD-1 toripalimab. The listed CDRs are
based on the Kabat numbering system.

| Name | Sequence | SEQ ID NO |
|---|---|---|
| LCDR1 | RSSQSIVHSNGNTYLE | SEQ ID NO: 11 |
| LCDR2 | KVSNRFS | SEQ ID NO: 12 |
| LCDR3 | FQGSHVPLT | SEQ ID NO: 13 |
| HCDR1 | DYEMH | SEQ ID NO: 14 |
| HCDR2 | VIESETGGTAYNQKFKG | SEQ ID NO: 15 |
| HCDR3 | EGITTVATTYYWYFDV | SEQ ID NO: 16 |
| VL | DVVMTQSPLSLPVTLGQPASISCRSSQSIVHSNGN | SEQ ID NO: 17 |
| | TYLEWYLQKPGQSPQLLIYKVSNRFSGVPDRFSG | |
| | SGSGTDFTLKISRVEAEDVGVYYCFQGSHVPLTF | |
| | GQGTKLEIK | |
| VH | QGQLVQSGAEVKKPGASVKVSCKASGYTFTDYE | SEQ ID NO: 18 |
| | MHWVRQAPIHGLEWIGVIESETGGTAYNQKFKG | |
| | RVTITADKSTSTAYMELSSLRSEDTAVYYCAREG | |
| | ITTVATTYYWYFDVWGQGTTVTVSS | |
| LC | DVVMTQSPLSLPVTLGQPASISCRSSQSIVHSNGN | SEQ ID NO: 19 |
| | TYLEWYLQKPGQSPQLLIYKVSNRFSGVPDRFSG | |
| | SGSGTDFTLKISRVEAEDVGVYYCFQGSHVPLTF | |
| | GQGTKLEIKRTVAAPSVFIFPPSDEQLKSGTASVV | |
| | CLLNNFYPREAKVQWKVDNALQSGNSQESVTEQ | |
| | DSKDSTYSLSSTLTLSKADYEKHKVYACEVTHQ | |
| | GLSSPVTKSFNRGEC | |
| HC | QGQLVQSGAEVKKPGASVKVSCKASGYTFTDYE | SEQ ID NO: 20 |
| | MHWVRQAPIHGLEWIGVIESETGGTAYNQKFKG | |
| | RVTITADKSTSTAYMELSSLRSEDTAVYYCAREG | |
| | ITTVATTYYWYFDVWGQGTTVTVSSASTKGPSV | |
| | FPLAPCSRSTSESTAALGCLVKDYFPEPVTVSWN | |
| | SGALTSGVHTFPAVLQSSGLYSLSSVVTVPSSSLG | |
| | TKTYTCNVDHKPSNTKVDKRVESKYGPPCPPCPA | |
| | PEFLGGPSVFLFPPKPKDTLMISRTPEVTCVVVDV | |
| | SQEDPEVQFNWYVDGVEVHNAKTKPREEQFNST | |
| | YRVVSVLTVLHQDWLNGKEYKCKVSNKGLPSSI | |
| | EKTISKAKGQPREPQVYTLPPSQEEMTKNQVSLT | |
| | CLVKGFYPSDIAVEWESNGQPENNYKTTPPVLDS | |
| | DGSFFLYSRLTVDKSRWQEGNVFSCSVMHEALH | |
| | NHYTQKSLSLSLGK | |

### Combination Treatment

In an aspect, an anti-BTLA combo-therapy comprises the use of anti-BLTA in combination with another immune checkpoint therapy. As used herein, the phrase “immune checkpoint therapy” refers to therapy with one or more agents capable of altering the function of immune checkpoints, including the CTLA-4, LAG-3, B7-H3, B7-H4, Tim3, BTLA, KIR, A2aR, CD200 and/or PD-1 pathways. Exemplary immune checkpoint modulating agents include anti-CTLA-4 antibody (e.g., ipilimumab), anti-LAG-3 antibody, anti-B7-H3 antibody, anti-B7-H4 antibody, anti-Tim3 antibody, anti-KIR antibody, anti-A2aR antibody, anti CD200 antibody, anti-PD-1 antibody, anti-PD-LI antibody, anti-CD28 antibody, anti-CD80 or -CD86 antibody, anti-B7RP1 antibody, anti-B7-H3 antibody, anti-HVEM antibody, anti-CD137 or -CD137L antibody, anti-OX40 or -OX40L antibody, anti-CD40 or -CD40L antibody, anti-GALS antibody, anti-IL-10 antibody and A2aR drug.

In an aspect, an anti-BTLA antibody or antigen binding fragment can be used alone or in combination with: other anti-neoplastic agents or immunogenic agents (for example, attenuated cancerous cells, tumor antigens (including recombinant proteins, peptides, and carbohydrate molecules), antigen presenting cells such as dendritic cells pulsed with tumor derived antigen or nucleic acids, immune stimulating cytokines (for example, IL-2, IFNa2, GM-CSF), and cells transfected with genes encoding immune stimulating cytokines such as but not limited to GM-CSF); standard cancer treatments (for example, chemotherapy, radiotherapy or surgery); or other antibodies (including but not limited to antibodies to PD-1, PD-L1, VEGF, EGFR, Her2/neu, VEGF receptors, other growth factor receptors, CD20, CD40, CTLA-4, OX-40, 4-1BB, and ICOS).

In an aspect, an anti-BTLA therapy is co-administered with another immune checkpoint therapy. “Co-administration” as used herein for agents such as the BTLA antagonist or PD-1 antagonist means that the agents are administered so as to have overlapping therapeutic activities, and not necessarily that the agents are administered simultaneously to the subject. The agents may or may not be in physical combination prior to administration. Co-administration includes, but is not limited to, sequential infusions with varying length of interval between two infusions.

In an aspect, a BTLA antagonist and a PD-1 or PD-L1 antagonist is co-formulated. “Co-formulated” or “co-formulation”, as used herein, refers to at least two different antibodies or antigen binding fragments thereof which are formulated together and stored as a combined product in a single vial or vessel (for example an injection device) rather than being formulated and stored individually and then mixed before administration or separately administered. In an aspect, the co-formulation contains two different antibodies or antigen binding fragments thereof.

In an aspect, an anti-BTLA antibody or antigen binding fragment is co-administered with a chemotherapeutic agent. “Chemotherapeutic agent” is a chemical compound useful in the treatment of cancer. Classes of chemotherapeutic agents include, but are not limited to: alkylating agents, antimetabolites, kinase inhibitors, spindle poison plant alkaloids, cytotoxic/antitumor antibiotics, topisomerase inhibitors, photosensitizers, anti -estrogens and selective estrogen receptor modulators (SERMs), anti-progesterones, estrogen receptor down-regulators (ERDs), estrogen receptor antagonists, leutinizing hormone-releasing hormone agonists, anti-androgens, aromatase inhibitors, EGFR inhibitors, VEGF inhibitors, anti-sense oligonucleotides or other small RNA molecules that that inhibit expression of genes implicated in abnormal cell proliferation or tumor growth. In an aspect, chemotherapeutic agents useful in the treatment methods of the present disclosure include cytostatic and/or cytotoxic agents.

In an aspect, an anti-BTLA antibody or antigen binding fragment is co-administered with a platinum-containing chemotherapy. “Platinum-containing chemotherapy” (also known as platins) refers to the use of chemotherapeutic agent(s) used to treat cancer that are coordination complexes of platinum. Platinum-containing chemotherapeutic agents are alkylating agents that crosslink DNA, resulting in ineffective DNA mismatch repair and generally leading to apoptosis. Examples of platins include cisplatin, carboplatin, and oxaliplatin.

In an aspect, an anti-BTLA mono- or combo-therapy (e.g., with an anti-PD-1 or anti-PD-L1) further comprises a chemotherapeutic agent. Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin gammall and calicheamicin phill, see, e.g., Agnew, Chem. Intl. Ed. Engl., 33: 183-186 (1994); dynemicin, including dynemicin A; bisphosphonates, such as clodronate; an esperamicin; as well as neocarzinostatin chromophore and related chromoprotein enediyne antibiotic chromomophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2 -ethylhydrazide; procarbazine; razoxane; rhizoxin; sizofuran; spirogermanium; tenuazonic acid; triaziquone; 2, 2′,2″-trichlorotriethylamine; trichothecenes(especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel and doxetaxel; chlorambucil; gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; vinorelbine ;novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above. Also included are anti-hormonal agents that act to regulate or inhibit hormone action on tumors such as anti estrogens and selective estrogen receptor modulators (SERMs), including, for example, tamoxifen, raloxifene, droloxifene, 4-hydroxytamoxifen, trioxifene, keoxifene, LY117018, onapristone, and toremifene (Fareston); aromatase inhibitors that inhibit the enzyme aromatase, which regulates estrogen production in the adrenal glands, such as, for example, 4(5)-imidazoles, aminoglutethimide, megestrol acetate, exemestane, formestane, fadrozole, vorozole, letrozole, and anastrozole; and anti-androgens such as flutamide, nilutamide, bicalutamide, leuprolide, and goserelin; and pharmaceutically acceptable salts, acids or derivatives of any of the above.

Each therapeutic agent in a combination therapy of the disclosure may be administered either alone or in a medicament (also referred to herein as a pharmaceutical composition) which comprises the therapeutic agent and one or more pharmaceutically acceptable carriers, excipients and diluents, according to standard pharmaceutical practice.

Each therapeutic agent in a combination therapy of the disclosure may be administered simultaneously (i.e., in the same medicament), concurrently (i.e., in separate medicaments administered one right after the other in any order) or sequentially in any order. Sequential administration is particularly useful when the therapeutic agents in the combination therapy are in different dosage forms (one agent is a tablet or capsule and another agent is a sterile liquid) and/or are administered on different dosing schedules, e.g., a chemotherapeutic that is administered at least daily and a biotherapeutic that is administered less frequently, such as once weekly, once every two weeks, or once every three weeks.

In an aspect, an anti-BTLA antibody is administered before administration of the anti-PD-1 antibody or anti-PD-L1 antibody, while in other aspects, the anti-BTLA antibody is administered after administration of the anti-PD-1 antibody or anti-PD-L1 antibody. In another aspect, the anti-BTLA antibody is administered concurrently with the anti-PD-1 antibody or anti-PD-L1 antibody.

In an aspect, at least one of the therapeutic agents in the combination therapy is administered using the same dosage regimen (dose, frequency and duration of treatment) that is typically employed when the agent is used as monotherapy for treating the same cancer. In other aspects, the patient receives a lower total amount of at least one of the therapeutic agents in the combination therapy than when the agent is used as monotherapy, e.g., smaller doses, less frequent doses, and/or shorter treatment duration.

Each small molecule therapeutic agent in a combination therapy of the disclosure can be administered orally or parenterally, including the intravenous, intramuscular, intraperitoneal, subcutaneous, rectal, topical, and transdermal routes of administration. A combination therapy of the disclosure may be used prior to or following surgery to remove a tumor and may be used prior to, during or after radiation therapy.

In an aspect, a combination therapy of the disclosure is administered to a patient who has not been previously treated with a biotherapeutic or chemotherapeutic agent, i.e., is treatment-naive. In other aspects, the combination therapy is administered to a patient who failed to achieve a sustained response after prior therapy with a biotherapeutic or chemotherapeutic agent, i.e., is treatment-experienced.

A combination therapy of the disclosure is typically used to treat a tumor that is large enough to be found by palpation or by imaging techniques well known in the art, such as MRI, ultrasound, or CAT scan. A combination therapy of the disclosure can be administered to a human patient who has a cancer that tests positive for one or both of PD-L1 and PD-L2, and preferably tests positive for PD-L1 expression. In some preferred aspects, PD-L1 expression is detected using a diagnostic anti-human PD-L1 antibody, or antigen binding fragment thereof, in an IHC assay on an FFPE or frozen tissue section of a tumor sample removed from the patient.

In another aspect, the anti-PD-1 antibody in the combination therapy is nivolumab, pembrolizumab, toripalimab, sintilimab, camrelizumab, tislelizumab, or cemiplimab, or (i) a variant or antigen binding fragment or (ii) a biosimilar molecule of any of the foregoing, which is administered in a liquid medicament at a dose selected from the group consisting of about 1 mg/kg Q2W, 2 mg/kg Q2W, 3 mg/kg Q2W, 5 mg/kg Q2W, 10 mg/kg Q2W, 1 mg/kg Q3W, 2 mg/kg Q3W, 3 mg/kg Q3W, 5 mg/kg Q3W, 10 mg/kg Q3W and flat-dose equivalents of any of these doses, i.e., such as about 200, 240, 280, 300 mg Q3W.

In another aspect, the anti-PD-1 antibody, or antigen binding fragment thereof, is administered to the patient once every four or six weeks for 12 weeks or more. In other aspects, the anti-PD-1 antibody, or antigen binding fragment thereof, is administered to the patient once every three or six weeks for 16 weeks or more, 18 weeks or more, 20 weeks or more, 24 weeks or more, 28 weeks or more, 30 weeks or more, 32 weeks or more, 36 weeks or more, 40 weeks or more, 42 weeks or more, 44 weeks or more, 48 weeks or more, 52 weeks or more, 54 weeks or more, 56 weeks or more, 60 weeks or more, 64 weeks or more, 66 weeks or more, 68 weeks or more, 72 weeks or more, 76 weeks or more, 78 weeks or more, 80 weeks or more, 84 weeks or more, 88 weeks or more, or 90 weeks or more.

In an aspect, a selected dose of an anti-BTLA or anti-PD-1/PD-L1 antibody is administered by IV infusion. In an aspect, the selected dose of an anti-BTLA or anti-PD-1/PD-L1 antibody is administered by IV infusion over a time period of between 25 and 60 minutes, or about 30 minutes.

In an aspect, the patient is treated with the combination therapy for at least 24 weeks, e.g., eight 3-week cycles. In an aspect, treatment with the combination therapy continues until the patient exhibits evidence of progressive disease or a complete response.

### Dose and Regimen

For the prevention or treatment of disease, the appropriate dosage of antibody in an anti-BTLA mono- or combo-treatment will depend on a variety of factors such as the type of disease to be treated, the severity and course of the disease, whether the antibody is administered for preventive or therapeutic purposes, previous therapy, the patient's clinical history and response to the antibody, and the discretion of the attending physician. An antibody is suitably administered to the patient at one time or over a series of treatments.

In an aspect, an anti-BTLA antibody or antigen binding fragment thereof is administered intravenously at a weight-based dose of 0.3-10 mg/kg Q3W, or at a flat dose of 20-500 mg Q3W. In anther aspect, an anti-BTLA antibody or antigen binding fragment thereof is administered intravenously at a weight-based dose of about 0.3, 0.5, 1, 2, 3, 4, 5, 6, 7, 8, 9 or 10 mg/kg Q3W. In anther aspect, an anti-BTLA antibody or antigen binding fragment thereof is administered intravenously at a flat dose of about 20, 30, 50, 75, 100, 150, 200, 250, 300, 350, 400, 450 or 500 mg Q3W.

In an aspect, an anti-BTLA antibody or antigen binding fragment thereof is administered intravenously at a weight-based dose of from about 0.3 to about 15, from about 0.3 to about 12, from about 0.3 to about 10, from about 0.3 to about 9, from about 0.3 to about 8, from about 0.3 to about 7, from about 0.3 to about 6, from about 0.3 to about 5, from about 0.3 to about 4, from about 0.3 to about 3, from about 0.3 to about 2, or from about 0.3 to about 1 mg/kg Q3W.

In another aspect, an anti-BTLA antibody or antigen binding fragment thereof is administered intravenously at a weight-based dose of from about 0.5 to about 15, from about 0.8 to about 12, from about 1 to about 10, from about 1.2 to about 8, from about 1.4 to about 6, from about 1.6 to about 5, from about 1.8 to about 4, or from about 2 to about 3.5 mg/kg Q3W.

In an aspect, an anti-BTLA antibody or antigen binding fragment thereof is administered intravenously at least once every 1, 2, 3, 4, 5, 6, 7, 8 or 9 weeks. In anther aspect, an anti-BTLA antibody or antigen binding fragment thereof is administered intravenously at most once every 1, 2, 3, 4, 5, 6, 7, 8 or 9 weeks.

In another aspect, an anti-BTLA antibody or antigen binding fragment thereof is administered intravenously at a flat dose of at least about 50, 100, 150., 200, 250, 300, 350, 400 or 450 mg at a frequency selected the group consisting of Q1W, Q2W, Q3W, Q4W, QSW, Q6W, Q7W, Q8W and Q9W.

In an aspect, an anti-BTLA antibody or antigen binding fragment thereof is administered intravenously at a flat dose of from about 20 to about 500, from about 30 to about 450, from about 50 to about 400, from about 75 to about 350, from about 100 to about 300, from about 125 to about 275, or from about 150 to about 250 mg at a frequency selected the group consisting of Q1W, Q2W, Q3W, Q4W, QSW, Q6W, Q7W, Q8W and Q9W.

In another aspect, an anti-BTLA antibody or antigen binding fragment thereof is administered intravenously at a flat dose of from about 20 to about 50, from about 50 to about 100, from about 100 to about 150, from about 150 to about 200, from about 200 to about 250, from about 250 to about 300, from about 300 to about 350, or from about 350 to about 400 mg Q3W.

In an aspect, an anti-BTLA antibody or antigen binding fragment thereof is administered intravenously at a weight-based dose of about 0.3, 1, 3, or 10 mg/kg Q3W, or at a flat dose of about 20, 70, 200 or 500 mg Q3W.

In another aspect, an anti-BTLA antibody or antigen binding fragment thereof is administered intravenously at a weight-based dose of about 3 mg/kg Q3W or at a flat dose of about 200 mg Q3W.

In an aspect, an anti-BTLA antibody or antigen binding fragment thereof is administered intravenously at a weight-based dose of about 3 mg/kg once every 1, 2, 3, 4, 5, 6, 7, 8 or 9 weeks, or at a flat dose of about 200 mg once every 1, 2, 3, 4, 5, 6, 7, 8 or 9 weeks.

In another aspect, an anti-BTLA antibody or antigen binding fragment thereof is administered for a treatment regimen lasting at least 3, 6, 9, 12, 16, 20, 24, 28, 32, 36, 42, 46, 50, 54, 58, 62 or 66 weeks or months.

In an aspect, an anti-PD-1 or anti-PD-L1 antibody or antigen binding fragment thereof is administered intravenously at a weight-based dose of 1-15 mg/kg Q3W, or at a flat dose of 120-500 mg Q3W. In anther aspect, an anti-PD-1 or anti-PD-L1 antibody or antigen binding fragment thereof is administered intravenously at a weight-based dose of about 1, 2, 3, 4, 5, 6, 7, 8, 9 or 10 mg/kg Q3W. In anther aspect, an anti-PD-1 or anti-PD-L1 antibody or antigen binding fragment thereof is administered intravenously at a flat dose of about 100, 120, 150, 200, 240, 250, 300, 350, 360, 400, 450, 480 or 500 mg Q3W.

In an aspect, an anti-PD-1 or anti-PD-L1 antibody or antigen binding fragment thereof is administered intravenously at a weight-based dose of from about 1 to about 15, from about 1 to about 12, from about 1 to about 10, from about 1 to about 9, from about 1 to about 8, from about 1 to about 7, from about 1 to about 6, from about 1 to about 5, from about 1 to about 4, from about 1 to about 3, or from about 1 to about 2 mg/kg Q3W.

In another aspect, an anti-PD-1 or anti-PD-L1 antibody or antigen binding fragment thereof is administered intravenously at a weight-based dose of from about 2 to about 15, from about 3 to about 12, from about 4 to about 10, from about 5 to about 8, from about 1 to about 6, from about 2 to about 5, from about 3 to about 4, or from about 3 to about 5 mg/kg Q3W.

In an aspect, an anti-PD-1 or anti-PD-L1 antibody or antigen binding fragment thereof is administered intravenously at least once every 1, 2, 3, 4, 5, 6, 7, 8 or 9 weeks. In anther aspect, an anti-PD-1 or anti-PD-v antibody or antigen binding fragment thereof is administered intravenously at most once every 1, 2, 3, 4, 5, 6, 7, 8 or 9 weeks.

In another aspect, an anti-PD-1 or anti-PD-L1 antibody or antigen binding fragment thereof is administered intravenously at a flat dose of at least about 100, 120, 150, 200, 240, 250, 300, 350, 360, 400, 450, 480 or 500 mg at a frequency selected the group consisting of Q1W, Q2W, Q3W, Q4W, Q5W, Q6W, Q7W, Q8W and Q9W.

In an aspect, an anti-PD-1 or anti-PD-L1 antibody or antigen binding fragment thereof is administered intravenously at a flat dose of from about 100 to about 500, from about 125 to about 450, from about 150 to about 400, from about 175 to about 350, from about 200 to about 300, from about 225 to about 275, or from about 150 to about 250 mg at a frequency selected the group consisting of Q1W, Q2W, Q3W, Q4W, Q5W, Q6W, Q7W, Q8W and Q9W.

In another aspect, an anti-PD-1 or anti-PD-L1 antibody or antigen binding fragment thereof is administered intravenously at a flat dose of from about 50 to about 100, from about 100 to about 150, from about 150 to about 200, from about 200 to about 250, from about 250 to about 300, from about 300 to about 350, or from about 350 to about 400 mg Q3W.

In an aspect, an anti-PD-1 or anti-PD-L1 antibody or antigen binding fragment thereof is administered intravenously at a weight-based dose of about 2, 3, 4, 6, 8 or 10 mg/kg Q3W, or at a flat dose of about 120, 240, 360 or 480 mg Q3W.

In another aspect, an anti-PD-1 or anti-PD-L1 antibody or antigen binding fragment thereof is administered intravenously at a weight-based dose of about 3 mg/kg Q3W or at a flat dose of about 240 mg Q3W.

In an aspect, an anti-PD-1 or anti-PD-L1 antibody or antigen binding fragment thereof is administered intravenously at a weight-based dose of about 3 mg/kg once every 1, 2, 3, 4, 5, 6, 7, 8 or 9 weeks, or at a flat dose of about 240 mg once every 1, 2, 3, 4, 5, 6, 7, 8 or 9 weeks.

In another aspect, an anti-PD-1 or anti-PD-L1 antibody or antigen binding fragment thereof is administered for a treatment regimen lasting at least 3, 6, 9, 12, 16, 20, 24, 28, 32, 36, 42, 46, 50, 54, 58, 62 or 66 weeks or months.

In an aspect, an anti-BTLA mono- or combo-therapy is administered to a patient until disease progression, disease recurrence, unacceptable toxicity, or up to about 12, 18, 24, 30, or 36 months.

### Administration

An anti-BTLA mono- or combo-therapy can be administered by any suitable means, including parenteral, subcutaneous, intraperitoneal, intrapulmonary, and intranasal, and, if desired for local immunosuppressive treatment, intralesional administration (including perfusing or otherwise contacting the graft with the antibody before transplantation). In an aspect, an anti-BTLA antibody or agent can be administered, for example, as an infusion or as a bolus. Parenteral infusions include intramuscular, intravenous, intraarterial, intraperitoneal, or subcutaneous administration. In another aspect, an anti-BTLA antibody is suitably administered by pulse infusion, particularly with declining doses of the antibody. In another aspect, the dosing is given by injections, most preferably intravenous or subcutaneous injections, depending in part on whether the administration is brief or chronic. In a further aspect, an anti-BTLA antibody or antigen-binding fragment thereof, or corresponding pharmaceutical compositions thereof described herein can be administrated by non-invasive routes (e.g., oral administration; e.g., oral administration in pills, capsules, or tablets) is also within the scope of the present invention. In another aspect, an anti-BTLA antibody or antigen-binding fragment thereof, or a pharmaceutical composition thereof is administered intramuscularly, intra-arterially, intra-articularly (e.g., in arthritic joints), by inhalation, by aerosol delivery, or intratumorally.

In an aspect, a single dose of an anti-BTLA, anti-PD-1 or anti-PD-L1 antibody or antigen binding fragment thereof is administered via intravenous infusion which lasts at least about 15, 30, 60, 90, 120 or 150 minutes.

In an aspect, a single dose of an anti-BTLA, anti-PD-1 or anti-PD-L1 antibody or antigen binding fragment thereof is administered via intravenous infusion which lasts at most about 15, 30, 60, 90, 120 or 150 minutes.

In an aspect, a single dose of an anti-BTLA, anti-PD-1 or anti-PD-L1 antibody or antigen binding fragment thereof is administered via intravenous infusion which lasts from 15 to 30, from 30 to 60, from 60 to 90, from 90 to 120, or from 120 to 150 minutes.

### Indications

The antibody or antigen binding fragments of the disclosure (e.g., anti-BTLA) can be used to treat cancer (i.e., to inhibit the growth or survival of tumor cells). Preferred cancers whose growth may be inhibited using the antibodies of the disclosure include cancers typically responsive to immunotherapy, but also cancers that have not hitherto been associated with immunotherapy. Non-limiting examples of preferred cancers for treatment include melanoma (e.g., metastatic malignant melanoma), squamous cell carcinoma of the head and neck, lymphoma, and other neoplastic malignancies. Additionally, the disclosure includes refractory or recurrent malignancies whose growth may be inhibited using the antibodies of the disclosure.

“Tumor” as it applies to a subject diagnosed with, or suspected of having, cancer refers to a malignant or potentially malignant neoplasm or tissue mass of any size, and includes primary tumors and secondary neoplasms. A solid tumor is an abnormal growth or mass of tissue that usually does not contain cysts or liquid areas. Different types of solid tumors are named for the type of cells that form them. Examples of solid tumors are sarcomas, carcinomas, and lymphomas. Leukemias (cancers of the blood) generally do not form solid tumors.

Cancers that may be treated by the antibodies, compositions and methods disclosed herein include, but are not limited to: Gastrointestinal: esophagus (squamous cell carcinoma, adenocarcinoma, leiomyosarcoma, lymphoma), large bowel (adenocarcinoma, tubular adenoma, villous adenoma, hamartoma, leiomyoma) colorectal; B-cell lymphoma, T-cell lymphoma, Hodgkins lymphoma, non-Hodgkins lymphoma, hairy cell lymphoma, mantle cell lymphoma, myeloma, and Burkett' s lymphoma; and other tumors, including melanoma, skin (non-melanomal) cancer, mesothelioma (cells), seminoma, teratocarcinoma, osteosarcoma, xenoderoma pigmentosum, keratoctanthoma, thyroid follicular cancer and Kaposi's sarcoma. In an aspect, the forgoing cancers are advanced, unresectable or metastatic. In an aspect, the patients are refractory to anti-PD-1 or anti-PD-L1 therapy.

In an aspect, cancers that may be treated by the antibodies, compositions and methods of the disclosure include, but are not limited to: colorectal cancer, melanoma, head and neck cancers, sarcomas, and non-Hodgkin's lymphoma,. In another aspect, cancers that may be treated by the antibodies, compositions and methods of the disclosure include, but are not limited to: head and neck squamous cell cancer, and colorectal cancer. In an aspect, the colorectal cancer is non-microsatellite instability-high (non-MSI-H) or proficient mismatch repair (pMMR). In an aspect, the patient with head and neck squamous cell cancer is anti-PD-1 or anti-PD-L1 treatment refractory. In an aspect, the colorectal cancer is unresectable or metastatic (Stage IV).

In another aspect, cancers that may be treated by the antibodies, compositions and methods of the disclosure include hematological malignancies, but are not limited to: classical Hodgkin lymphoma (cHL), diffuse large B-cell lymphoma (DLBCL), transformed DLBCL, gray zone lymphoma, double hit lymphoma, Primary mediastinal B cell lymphoma (PMBCL) or indolent non-Hodgkin lymphoma (iNHL) (for example, follicular lymphoma, marginal zone lymphoma, mucosa-associated lymphoid tissue lymphoma, or small lymphocytic lymphoma). In an aspect, the patient with Hodgkin lymphoma is anti-PD-1 or anti-PD-L1 treatment refractory.

In an aspect, a cancer being treated herein is an advanced cancer. “Advanced” cancer is one which has spread outside the site or organ of origin, either by local invasion or metastasis. Accordingly, the term “advanced” cancer includes both locally advanced and metastatic disease.

In an aspect, a cancer being treated herein is a recurrent cancer or a locally recurrent cancer. “Recurrent” cancer is one which has regrown, either at the initial site or at a distant site, after a response to initial therapy, such as surgery. A “locally recurrent” cancer is cancer that returns after treatment in the same place as a previously treated cancer.

In an aspect, a cancer being treated herein is an unresectable cancer. “Unresectable” cancer is not able to be removed (resected) by surgery.

In an aspect, a cancer being treated herein is a metastatic cancer. “Metastatic” cancer refers to cancer which has spread from one part of the body (e.g. the lung) to another part of the body.

In an aspect, a cancer being treated herein is a locally advanced cancer. “Locally advanced” cancer refers to cancer that has spread to nearby tissues or lymph nodes, but not metastasized.

In an aspect, a cancer being treated herein is an advanced unresectable cancer. “Advanced unresectable” cancer is one which has spread outside the site or organ of origin, either by local invasion or metastasis and which is not able to be removed (resected) by surgery.

In an aspect, an anti-BTLA therapy disclosed herein is for the treatment of patients with melanoma, preferably unresectable or metastatic melanoma. In another aspect, an anti-BTLA therapy disclosed herein is for the adjuvant treatment of adult and pediatric (12 years and older) patients with Stage IIB, IIC, or III melanoma following complete resection. In another aspect, the melanoma is advanced or Stage III. In an aspect, the melanoma patients are refractory to anti-PD-1 or anti-PD-L1 therapy.

In an aspect, an anti-BTLA therapy disclosed herein is for treating Head and Neck Squamous Cell Cancer (HNSCC). In another aspect, an anti-BTLA therapy is in combination with platinum and FU for the first-line treatment of patients with metastatic or with unresectable, recurrent HNSCC. In an aspect, an anti-BTLA therapy disclosed herein is used as a single agent for the first-line treatment of patients with metastatic or with unresectable, recurrent HNSCC whose tumors express PD-L1 (e.g., Combined Positive Score (CPS)≥1, where Combined Positive Score (CPS) refers to the number of PD-L1 staining cells (tumor cells, lymphocytes, macrophages) divided by the total number of viable tumor cells, multiplied by 100.). In an aspect, an anti-BTLA therapy disclosed herein is used as a single agent for the treatment of patients with recurrent or metastatic HNSCC with disease progression on or after platinum-containing chemotherapy.

In an aspect, an anti-BTLA therapy disclosed herein is for treating Classical Hodgkin Lymphoma (cHL). In another aspect, an anti-BTLA therapy is for the treatment of adult patients with relapsed or refractory cHL. In another aspect, an anti-BTLA therapy is for the treatment of pediatric patients with refractory cHL, or cHL that has relapsed after 2 or more lines of therapy.

In an aspect, an anti-BTLA therapy disclosed herein is for treating Primary Mediastinal Large B-Cell Lymphoma (PMBCL). In another aspect, an anti-BTLA therapy is for the treatment of adult and pediatric patients with refractory PMBCL, or who have relapsed after 2 or more prior lines of therapy.

In an aspect, an anti-BTLA therapy disclosed herein is for the treatment of adult and pediatric patients with unresectable or metastatic, microsatellite instability-high (MSI-H) or mismatch repair deficient (dMMR) solid tumors that have progressed following prior treatment and/or who have no satisfactory alternative treatment options. In an aspect, an anti-BTLA therapy disclosed herein is for the treatment of patients with unresectable or metastatic MSI-H or dMMR colorectal cancer (CRC).

### Treatment Effects

In an aspect, cancer treatment effects are assessed using “RECIST 1.1 Response Criteria” as set forth in Eisenhauer et al., Eur. J Cancer 45:228-247 (2009) for target lesions or nontarget lesions, as appropriate based on the context in which response is being measured.

In an aspect, cancer treatment effects are assessed using “Unidimensional irRC” as described in Nishino et al., Developing a Common Language for Tumor Response to Immunotherapy: Immune-related Response Criteria using Unidimensional measurements. Clin Cancer Res.19(14):3936-3943) (2013). These criteria utilize the longest diameter (cm) of each lesion.

In an aspect, a therapy or treatment described here induces an anti-tumor response in a patient. “Anti-tumor response” when referring to a cancer patient treated with a therapeutic regimen, such as an anti-BTLA therapy or anti-BTLA/anti-PD-(L)1 combination therapy described herein, means at least one positive therapeutic effect, such as for example, reduced number of cancer cells, reduced tumor size, reduced rate of cancer cell infiltration into peripheral organs, reduced rate of tumor metastasis or tumor growth, or progression free survival. Positive therapeutic effects in cancer can be measured in a number of ways (See Weber, J. Nucl. Med. 50: 1S-10S (2009)).

In an aspect, the treatment achieved by a therapeutically effective amount is any of progression free survival (PFS), disease free survival (DFS) or overall survival (OS). PFS, also referred to as “Time to Tumor Progression” indicates the length of time during and after treatment that the cancer does not grow, and includes the amount of time patients have experienced a complete response or a partial response, as well as the amount of time patients have experienced stable disease. DFS refers to the length of time during and after treatment that the patient remains free of disease. OS refers to a prolongation in life expectancy as compared to naive or untreated individuals or patients.

In an aspect, an anti-tumor response to a therapy described herein is assessed using RECIST 1.1 criteria, bidimensional irRC or unidimensional irRC. In an aspect, an anti-tumor response is any of SD, PR, CR, PFS, or DFS. “Bidimensional irRC” refers to the set of criteria described in Wolchok J D et al., Guidelines for the evaluation of immune therapy activity in solid tumors: immune-related response criteria. Clin Cancer Res.15(23):7412-7420 (2009). These criteria utilize bidimensional tumor measurements of target lesions, which are obtained by multiplying the longest diameter and the longest perpendicular diameter (cm²) of each lesion.

While an aspect of the treatment methods, compositions and uses of the present disclosure may not be effective in achieving a positive therapeutic effect in every patient, it should do so in a statistically significant number of subjects as determined by any statistical test known in the art such as the Student's t-test, the chi²-test, the U-test according to Mann and Whitney, the Kruskal-Wallis test (H-test), Jonckheere-Terpstra-test and the Wilcoxon-test.

In another aspect, a therapy disclosed herein reduces tumor burden in a patient. “Tumor burden” also referred to as “tumor load”, refers to the total amount of tumor material distributed throughout the body. Tumor burden refers to the total number of cancer cells or the total size of tumor(s), throughout the body, including lymph nodes and bone marrow. Tumor burden can be determined by a variety of methods known in the art, such as, e.g. by measuring the dimensions of tumor(s) upon removal from the subject, e.g., using calipers, or while in the body using imaging techniques, e.g., ultrasound, bone scan, computed tomography (CT) or magnetic resonance imaging (MRI) scans.

In another aspect, a therapy disclosed herein reduces tumor size in a patient. The term “tumor size” refers to the total size of the tumor which can be measured as the length and width of a tumor. Tumor size may be determined by a variety of methods known in the art, such as, e.g. by measuring the dimensions of tumor(s) upon removal from the subject, e.g., using calipers, or while in the body using imaging techniques, e.g., bone scan, ultrasound, CT or MRI scans.

In an aspect, an anti-BTLA mono- or combo-therapy (e.g., with anti-PD-1 or anti-PD-L1) described herein achieves a sustained response. “Sustained response” means a sustained therapeutic effect after cessation of treatment with a therapeutic agent, or a combination therapy described herein. In an aspect, the sustained response has a duration that is at least the same as the treatment duration, or at least 1.5, 2.0, 2.5 or 3 times longer than the treatment duration.

In an aspect, an anti-BTLA mono- or combo-therapy (e.g., with anti-PD-1 or anti-PD-L1) described herein achieves an objective response rate (ORR) of at least 10%, 15%, 20%, 25%, 30%, 35%, 40%, 45%, 50%, 55%, 60%, 65%, 70%, 80%, 90% or 95%. In another aspect, an anti-BTLA mono- or combo-therapy achieves an objective response rate (ORR) of between 10% and 15%, between 15% and 20%, between 20% and 25%, between 25% and 30%, between 30% and 35%, between 35% and 40%, between 40% and 45%, between 45% and 50%, between 50% and 55%, between 55% and 60%, between 60% and 65%, between 65% and 70%, between 75% and 80%, between 85% and 90% or between 90% and 95%. In another aspect, the foregoing ORR is achieved within 3, 4, 5, 6, 7, 8, 9, 10, 11, 12, 13, 14, 15, 16, 17, 18, 19, 20, 21, 22, 23 or 24 months from the first dosing. In another aspect, the foregoing ORR is assessed or assessable using one of more of the following criteria systems: RECIST, RECIST 1.1, Unidimensional Immune-related Response Criteria (irRC), and bidimensional irRC.

As used herein, “objective response rate” refers to the percentage of patients achieving an objection response. “Objective response” refers to a measurable response, including complete response (CR) or partial response (PR). “Complete response” or “complete remission” refers to the disappearance of all signs of cancer in response to treatment. This does not always mean the cancer has been cured. “Partial response” refers to a decrease in the size of one or more tumors or lesions, or in the extent of cancer in the body, in response to treatment.

### Biomarker/Patient Populations

In an aspect, a cancer being treated is PD-L1 positive. A “PD-L1 positive” cancer is one comprising cells which have PD-L1 present at their cell surface. Preferably, the cancer is “PD-L1 positive” according to the disclosure, when between at least 0.1% and at least 10% of the cells of the cancer have PD-L1 present at their cell surface. More preferably, the cancer is “PD-L1 positive”, when between at least 0.5% and 5% of the cells of the cancer have PD-L1 present at their cell surface. Most preferably, the cancer is “PD-L1 positive”, when at least 1%, 2%, 5%, 10%, 20%, 25%, 30%, 40%, 50%, 60% or 70% of the cells of the cancer have PD-L1 present at their cell surface.

The term “PD-L1 positive” also refers to a cancer that produces sufficient levels of PD-L1 at the surface of cells thereof, such that an anti-PD-L1 inhibitor (e.g. antibody) has a therapeutic effect, mediated by the binding of the said anti-PD-v inhibitor (e.g. antibody) to PD-L1.

“PD-L1” or “PD-L2” expression means any detectable level of expression of the designated PD-L protein on the cell surface or of the designated PD-L mRNA within a cell or tissue, unless otherwise defined. PD-L protein expression may be detected with a diagnostic PD-L antibody in an immunohistochemistry (IHC) assay of a tumor tissue section or by flow cytometry. Alternatively, PD-L protein expression by tumor cells may be detected by PET imaging, using a binding agent (e.g., antibody fragment, affibody and the like) that specifically binds to the desired PD-L target, e.g., PD-L1 or PD-L2. Techniques for detecting and measuring PD-L mRNA expression include RT-PCR and real-time quantitative RT-PCR.

Similarly, HVEM expression means any detectable level of expression of the HVEM protein on the cell surface or of the HVEM mRNA within a cell or tissue, unless otherwise defined. HVEM protein expression may be detected with a diagnostic HVEM antibody in an IHC assay of a tumor tissue section or by flow cytometry. Alternatively, HVEM protein expression by tumor cells may be detected by PET imaging, using a binding agent (e.g., antibody fragment, affibody and the like) that specifically binds to HVEM. Techniques for detecting and measuring HVEM mRNA expression include RT-PCR and real-time quantitative RT-PCR.

Several approaches have been described for quantifying PD-L1 protein expression in IHC assays of tumor tissue sections. See, e.g., Thompson et al., PNAS 101 (49): 17174-17179 (2004); Thompson et al., Cancer Res. 66:3381-3385 (2006); Gadiot et al., Cancer 117:2192- 2201 (2011); Taube et al., Sci Transl Med 4, 127ra37 (2012); and Toplian et al., New Eng. J Med. 366 (26): 2443-2454 (2012). Similar methods can be adopted for quantifying HVEM expression in tumor tissues. See also, Inoue et al., Anticancer Research 35(3):1361-1367 (2015); Malissen et al., Oncoimmunology 8(12): e1665976 (2019); Fang, etal., Journal of BUON 22(1):80-86 (2017); Ren et al., Lung Cancer 125:115-20 (2018). Similarly, several methods can be adopted for quantifying CD8 expression in tumor tissues. See, e.g., Si et al. BMC Cancer 22:211 (2022); Cal et al. Frontiers in Immunology August 2021, Volume 12, Article 704965.

In an aspect, the expression of a protein biomarker is determined using Tumor Proportion Score (TPS) which refers to the percentage of viable tumor cells showing partial or complete membrane staining at any intensity. For PD-L1 expression, various immunohistochemistry assays (e.g., 28-8, 22C3, SP263, and SP142) can be used. See Prince etal., JCO Precision Oncology no. 5 953-973 (2021). In an aspect, a tumor specimen is considered to have PD-L1 expression if TPS≥1% and high PD-L1 expression if TPS≥50%. Unless specified otherwise, the quantification of biomarker expression (e.g., PD-L1, HVEM, or CD8) disclosed in this application can be conducted following an assay referenced herein, or alternatively, following a comparable assay that would be appropriate for the biomarker expression threshold disclosed herein.

In an aspect, a cancer patient treated herein exhibits high frequency microsatellite instability. In another aspect, a cancer patient treated herein exhibits low frequency microsatellite instability. In another aspect, a cancer patient treated herein exhibits microsatellite Stable. As used herein, “microsatellite instability (MSI)” refers to the form of genomic instability associated with defective DNA mismatch repair in tumors. See Boland et al., Cancer Research 58, 5258-5257, (1998). In an aspect, MSI analysis can be carried out using the five National Cancer Institute (NCI) recommended microsatellite markers BAT25 (GenBank accession no. 9834508), BAT26 (GenBank accession no. 9834505), D5S346 (GenBank accession no. 181171), D2S123 (GenBank accession no. 187953), D17S250 (GenBank accession no. 177030). Additional markers for example, BAT40, BAT34C4, TGF-P-RII and ACTC can be used. Commercially available kits for MSI analysis include, for example, the Promega MSI multiplex PCR assay. As used herein, “high frequency microsatellite instability” or “microsatellite instability-high (MSI-H)” refers to if two or more of the five NCI markers show instability or >30-40% of the total markers demonstrate instability (i.e. have insertion/deletion mutations). “Low frequency microsatellite instability” or “microsatellite instability-low (MSI-L)” refers to if one of the five NCI markers show instability or <30-40% of the total markers exhibit instability (i.e. have insertion/deletion mutations). “Microsatellite Stable (MSS)” refers to if none of the five NCI markers show instability (i.e. have insertion/deletion mutations).

### Formulations

In an aspect, an anti-BTLA antibody or antigen binding fragment thereof used herein is in a formulation described in WO02021147846.

In an aspect, an anti-BTLA antibody or antigen binding fragment thereof is in a formulation comprising 10-30 mM histidine buffer, 30-100 mM sodium chloride, 50-200 mM trehalose, 0.01%-0.05% polysorbate 80, 10-50 mg/ml anti-BTLA antibody or antigen binding fragment thereof, wherein the histidine buffer comprises a molar ratio of 1:2, 1:3, 1:4 or 1:5 for histidine to histidine hydrochloride.

In another aspect, an anti-BTLA antibody or antigen binding fragment thereof is in a formulation comprising comprises about 20 mM histidine buffer, about 50 mM sodium chloride, about 140 mM trehalose, about 0.02% polysorbate 80, about 20 mg/ml anti-BTLA antibody or antigen binding fragment thereof, wherein the histidine buffer comprises a molar ratio of 1:3 for histidine to histidine hydrochloride.

Furthermore, a therapeutic agent disclosed herein can be in various formulations. Pharmaceutically acceptable excipients of the present disclosure include for instance, solvents, bulking agents, buffering agents, tonicity adjusting agents, and preservatives (see, e.g., Pramanick et al., Pharma Times, 45:65-77 (2013)). In an aspect the pharmaceutical compositions may comprise an excipient that functions as one or more of a solvent, a bulking agent, a buffering agent, and a tonicity adjusting agent (e.g., sodium chloride in saline may serve as both an aqueous vehicle and a tonicity adjusting agent). The pharmaceutical compositions of the present disclosure are suitable for parenteral administration.

In an aspect, the pharmaceutical compositions comprise an aqueous vehicle as a solvent. Suitable vehicles include for instance sterile water, saline solution, phosphate buffered saline, and Ringer's solution. In an aspect, the composition is isotonic.

The pharmaceutical compositions may comprise a bulking agent. Bulking agents are particularly useful when the pharmaceutical composition is to be lyophilized before administration. In an aspect, the bulking agent is a protectant that aids in the stabilization and prevention of degradation of the active agents during freeze or spray drying and/or during storage. Suitable bulking agents are sugars (mono-, di- and polysaccharides) such as sucrose, lactose, trehalose, mannitol, sorbital, glucose and raffinose.

The pharmaceutical compositions may comprise a buffering agent. Buffering agents control pH to inhibit degradation of the active agent during processing, storage and optionally reconstitution. Suitable buffers include for instance salts comprising acetate, citrate, phosphate or sulfate. Other suitable buffers include for instance amino acids such as arginine, glycine, histidine, and lysine. The buffering agent may further comprise hydrochloric acid or sodium hydroxide. In an aspect, the buffering agent maintains the pH of the composition within a range of 4 to 9. In an aspect, the pH is greater than (lower limit) 4, 5, 6, 7 or 8. In an aspect, the pH is less than (upper limit) 9, 8, 7, 6 or 5. That is, the pH is in the range of from about 4 to 9 in which the lower limit is less than the upper limit.

The pharmaceutical compositions may comprise a tonicity adjusting agent. Suitable tonicity adjusting agents include for instance dextrose, glycerol, sodium chloride, glycerin and mannitol.

The pharmaceutical compositions may comprise a preservative. Suitable preservatives include for instance antioxidants and antimicrobial agents. However, in preferred aspects, the pharmaceutical composition is prepared under sterile conditions and is in a single use container, and thus does not necessitate inclusion of a preservative.

In an aspect, a medicament comprising an anti-BTLA or anti-PD-1 antibody may be provided as a liquid formulation or a lyophilized formulation.

In an aspect, medicaments described herein may be provided as a kit which comprises a first container and a second container and a package insert. The first container contains at least one dose of a medicament comprising a PD-1 antagonist, the second container contains a medicament comprising the anti-BTLA antibody, and the package insert, or label, which comprises instructions for treating a patient for cancer using the medicaments. The first and second containers may be comprised of the same or different shape (e.g., vials, syringes and bottles) and/or material (e.g., plastic or glass). The kit may further comprise other materials that may be useful in administering the medicaments, such as diluents, filters, IV bags and lines, needles and syringes. In some preferred aspects of the kit, the PD-1 antagonist is an anti-PD-1 antibody and the instructions state that the medicaments are intended for use in treating a patient having cancer that tests positive for PD-L1 expression by an IHC assay.

A variety of further modifications and improvements in and to the methods of the present disclosure will be apparent to those skilled in the art. The following non-limiting embodiments are specifically envisioned:
1. A method for treating a solid tumor in a patient in need thereof, the method comprising: administering to the patient a pharmaceutically effective amount of an anti-BTLA antibody or antigen binding fragment thereof.
2. The method of embodiment 1, wherein the solid tumor is selected from the group consisting of colorectal cancer, classic Hodgkin's lymphoma (cHL), non-Hodgkin's lymphoma (NHL), B cell lymphoma, head and neck cancer, melanoma, germinal-center B-cell-like (GCB) diffuse large B-cell lymphoma (DLBCL), activated B-cell-like (ABC) DLBCL, follicular lymphoma (FL), mantle cell lymphoma (MCL), marginal zone lymphoma (MZL), lymphoplasmacytic lymphoma (LL), Waldenstrom macroglobulinemia (WM), central nervous system lymphoma (CNSL), Burkitt's lymphoma (BL), Splenic marginal zone lymphoma, Splenic lymphoma, unclassifiable, Splenic diffuse red pulp small B-cell lymphoma, Extranodal marginal zone lymphoma of mucosa-associated lymphoid tissue (MALT lymphoma), Nodal marginal zone lymphoma, Pediatric nodal marginal zone lymphoma, Pediatric follicular lymphoma, Primary cutaneous follicle centre lymphoma, T-cell/histiocyte rich large B-cell lymphoma, Primary DLBCL of the CNS, Primary cutaneous DLBCL, leg type, EBV-positive DLBCL of the elderly, DLBCL associated with chronic inflammation, Primary mediastinal (thymic) large B-cell lymphoma, Intravascular large B-cell lymphoma, ALK-positive large B-cell lymphoma, Plasmablastic lymphoma, Large B-cell lymphoma arising in HHV8-associated multicentric Castleman disease, Primary effusion lymphoma, B-cell lymphoma, unclassifiable, with features intermediate between diffuse large B-cell lymphoma and Burkitt lymphoma, and B-cell lymphoma, unclassifiable, with features intermediate between diffuse large B-cell lymphoma and classical Hodgkin lymphoma.
3. The embodiment of claim 1, wherein the solid tumor is selected from the group consisting of melanoma, colorectal cancer (CRC), sarcoma, neuroendocrine tumor (NET), squamous cell carcinoma (SCC) of the parotid gland, head and neck squamous cell carcinomas (HNSCCs), follicular lymphoma, Hodgkin's lymphoma, diffuse large B cell lymphoma (DLBCL).
4. The method of any one of embodiments 1 to 3, wherein the solid tumor is an advanced solid tumor malignancy.
5. The method of embodiment 1, wherein the solid tumor is one or more selected from the group consisting of a carcinoma, squamous carcinoma, adenocarcinoma, sarcoma, neuroma, melanoma, and lymphoma.
6. The method of embodiment 5, wherein the squamous carcinoma is in the eyelid, tunica conjunctiva, vagina, oral cavity, skin, urinary bladder, tongue, larynx, or gullet.
7. The method of embodiment 5, wherein the adenocarcinoma is in the prostate, small intestine, endometrium, large intestine, pancreas, gullet, intestinum rectum, uterus, mammary gland, or ovary.
8. The method of any one of embodiments 1 to 7, the solid tumor expresses HVEM.
9. The method of claim 8, wherein at least about 1%, 5%, 10%, 15%, 20%, 30%, 40%, 50%, 60%, 70%, 80%, 90%, or 95% of tumor cells are positive for HVEM.

10. The method of any one of embodiments 1 to 7, the solid tumor co-expresses HVEM and CD8.
11. The method of embodiment 9, wherein at least about 1%, 5%, 10%, 15%, 20%, 30%, 40%, 50%, 60%, 70%, 80%, 90%, or 95% of tumor cells are positive for HVEM and CD8.
12. The method of any one of embodiments 1 to 11, wherein the treatment with the anti-BTLA antibody or antigen binding fragment thereof is used as an adjuvant therapy.
13. The method of any one of embodiments 1 to 11, wherein the patient receives at least one, two, three or four prior lines of therapy.
14. The method of any one of embodiments 1 to 11, wherein the patient receives at least one prior line of chemotherapy and/or immune-therapy.
15. The method of any one of embodiments 1 to 11, wherein the patient receives at least one prior line of therapy selected from the group consisting of chemotherapy, radiotherapy, surgery, adjuvant therapy, molecular targeted drugs and immune checkpoint inhibitors.
16. The method of any one of embodiments 1 to 11, wherein the pharmaceutically effective amount of the anti-BTLA antibody or antigen binding fragment thereof is administered to the patient in combination with a pharmaceutically effective amount of one or more chemotherapy drugs.
17. The method of embodiment 16, wherein the one or more chemotherapy drugs are selected from the group consisting of an alkylating agent, a nitrosourea agent, an antimetabolite, an antitumor antibiotic, an alkaloid derived from a plant, a topoisomerase inhibitor, a hormone therapy medicine, a hormone antagonist, an aromatase inhibitor, a P-glycoprotein inhibitor and a platinum complex derivative.
18. The method of any one of embodiments 1 to 17, wherein the pharmaceutically effective amount of the anti-BTLA antibody or antigen binding fragment thereof achieves a BTLA receptor occupancy (RO) rate of about 80% or higher within about 1, 2, 3, 4, 5, 6, 7 or 8 days post administration.
19. The method of any one of embodiments 1 to 17, wherein the pharmaceutically effective amount of the anti-BTLA antibody or antigen binding fragment thereof achieves a BTLA receptor occupancy (RO) rate of about 60%, 70%, 80%, 90%, 95%, 98%, or 99% or higher within about 1, 2, 3, 4, 5, 6, 7 or 8 days post administration.
20. The method of any one of embodiments 1 to 17, wherein the pharmaceutically effective amount of the anti-BTLA antibody or antigen binding fragment thereof achieves a sustained BTLA receptor occupancy (RO) rate of about 80% or higher for at least about 1, 2, 3, 4, 5, 6, 7 or 8 weeks post administration.
21. The method of any one of embodiments 1 to 17, wherein the pharmaceutically effective amount of the anti-BTLA antibody or antigen binding fragment thereof achieves a sustained BTLA receptor occupancy (RO) rate of about 60%, 70%, 80%, 90%, 95%, 98%, 99% or higher for at least about 1, 2, 3, 4, 5, 6, 7 or 8 weeks post administration.
22. The method of any one of embodiments 1 to 17, wherein the pharmaceutically effective amount of the anti-BTLA antibody or antigen binding fragment thereof achieves a substantially complete BTLA receptor occupancy (RO).
23. The method of any one of embodiments 1 to 21, wherein tumor proliferation is suppressed.
24. The method of any one of embodiments 1 to 21, wherein tumor metastasis is suppressed.
25. The method of any one of embodiments 1 to 21, wherein the method results in a complete response.

26. The method of any one of embodiments 1 to 21, wherein the method results in a partial response.
27. The method of any one of embodiments 1 to 21, wherein the method stabilizes the condition of the solid tumor.
28. The method of any one of embodiments 1 to 21, wherein the method achieves a duration of response of at least about 1, 2, 3, 4, 5, 6, 7, 8, 9, 10, 12, 16, 18, 20, 24, 28, 32, 36, 40, 44, or 48 weeks.
29. The method of any one of embodiments 1 to 28, wherein the anti-BTLA antibody or antigen binding fragment thereof is administered as an intravenous infusion.
30. The method of any one of embodiments 1 to 29, wherein the anti-BTLA antibody or antigen binding fragment thereof is administered intravenously at a weight-based dose of 0.3-10 mg/kg Q3W, or at a flat dose of 20-500 mg Q3W.
31. The method of any one of embodiments 1 to 29, wherein the anti-BTLA antibody or antigen binding fragment thereof is administered intravenously at a weight-based dose of about 0.3, 0.5, 1, 2, 3, 4, 5, 6, 7, 8, 9 or 10 mg/kg Q3W.
32. The method of any one of embodiments 1 to 29, wherein the anti-BTLA antibody or antigen binding fragment thereof is administered intravenously at a flat dose of about 20, 30, 50, 75, 100, 150, 200, 250, 300, 350, 400, 450 or 500 mg Q3W.
33. The method of any one of embodiments 1 to 29, wherein the anti-BTLA antibody or antigen binding fragment thereof is administered intravenously at a weight-based dose of from about 0.3 to about 15, from about 0.3 to about 12, from about 0.3 to about 10, from about 0.3 to about 9, from about 0.3 to about 8, from about 0.3 to about 7, from about 0.3 to about 6, from about 0.3 to about 5, from about 0.3 to about 4, from about 0.3 to about 3, from about 0.3 to about 2, or from about 0.3 to about 1 mg/kg Q3W.
34. The method of any one of embodiments 1 to 29, wherein the anti-BTLA antibody or antigen binding fragment thereof is administered intravenously at a weight-based dose of from about 0.5 to about 15, from about 0.8 to about 12, from about 1 to about 10, from about 1.2 to about 8, from about 1.4 to about 6, from about 1.6 to about 5, from about 1.8 to about 4, or from about 2 to about 3.5 mg/kg Q3W.
35. The method of any one of embodiments 1 to 29, wherein the anti-BTLA antibody or antigen binding fragment thereof is administered intravenously at least once every 1, 2, 3, 4, 5, 6, 7, 8 or 9 weeks.
36. The method of any one of embodiments 1 to 29, wherein the anti-BTLA antibody or antigen binding fragment thereof is administered intravenously at most once every 1, 2, 3, 4, 5, 6, 7, 8 or 9 weeks.
37. The method of any one of embodiments 1 to 29, wherein the anti-BTLA antibody or antigen binding fragment thereof is administered intravenously at a flat dose of at least about 50, 100, 150., 200, 250, 300, 350, 400 or 450 mg at a frequency selected the group consisting of Q1W, Q2W, Q3W, Q4W, QSW, Q6W, Q7W, Q8W and Q9W.
38. The method of any one of embodiments 1 to 29, wherein the anti-BTLA antibody or antigen binding fragment thereof is administered intravenously at a flat dose of from about 20 to about 500, from about 30 to about 450, from about 50 to about 400, from about 75 to about 350, from about 100 to about 300, from about 125 to about 275, or from about 150 to about 250 mg Q3W.
39. The method of any one of embodiments 1 to 29, wherein the anti-BTLA antibody or antigen binding fragment thereof is administered intravenously at a flat dose of from about 20 to about 50, from about 50 to about 100, from about 100 to about 150, from about 150 to about 200, from about 200 to about 250, from about 250 to about 300, from about 300 to about 350, or from about 350 to about 400 mg Q3W.
40. The method of any one of embodiments 1 to 29, wherein the anti-BTLA antibody or antigen binding fragment thereof is administered intravenously at a weight-based dose of about 0.3, 1, 3, or 10 mg/kg Q3W, or at a flat dose of about 20, 70, 200 or 500 mg Q3W.
41. The method of any one of embodiments 1 to 29, wherein the anti-BTLA antibody or antigen binding fragment thereof is administered intravenously at a weight-based dose of about 3 mg/kg Q3W or at a flat dose of about 200 mg Q3W.
42. The method of any one of embodiments 1 to 29, wherein the anti-BTLA antibody or antigen binding fragment thereof is administered intravenously at a weight-based dose of about 3 mg/kg once every 1, 2, 3, 4, 5, 6, 7, 8 or 9 weeks, or at a flat dose of about 200 mg once every 1, 2, 3, 4, 5, 6, 7, 8 or 9 weeks.
43. The method of any one of embodiments 1 to 29, wherein the anti-BTLA antibody or antigen binding fragment thereof is administered at a dose of about 3 mg/kg.
44. The method of any one of embodiments 1 to 43, wherein the anti-BTLA antibody or antigen binding fragment thereof is administered for a treatment regimen lasting at least 3, 6, 9, 12, 16, 20, 24, 28, 32, 36, 42, 46, 50, 54, 58, 62 or 66 weeks or months.
45. The method of any one of embodiments 1 to 44, wherein the anti-BTLA antibody binds to human BTLA and is a chimeric, humanized, or human antibody.
46. The method of any one of embodiments 1 to 44, wherein the anti-BTLA antibody is a domain antibody or a bivalent antibody.
47. The method of any one of embodiments 1 to 44, wherein the anti-BTLA antigen binding fragment is selected from the group consisting of Fab, Fab′, F(ab′)2, Fv, scFv and sdAb.
48. The method of any one of embodiments 1 to 45, wherein the anti-BTLA antibody comprises a heavy chain constant region of an IgG1, IgG2, IgG3, or IgG4 isotype.
49. The method of any one of embodiments 1 to 48, wherein the anti-BTLA antibody or antigen binding fragment thereof comprises a serine 227 to proline substitution in the hinge region of the heavy chain.
50. The method of any one of embodiments 1 to 49, wherein the anti-BTLA antibody or antigen binding fragment thereof is glycosylated.
51. The method of any one of embodiments 1 to 50, wherein the anti-BTLA antibody or antigen binding fragment thereof comprises a light chain variable region of LCDR1 having the amino acid sequence of SEQ ID NO: 1, LCDR2 having the amino acid sequence of SEQ ID NO: 2, LCDR3 having the amino acid sequence of SEQ ID NO: 3; and a heavy chain variable region of HCDR1 having the amino acid sequence of SEQ ID NO: 4, HCDR2 having the amino acid sequence of SEQ ID NO: 5, HCDR3 having the amino acid sequence of SEQ ID NO:6.
52. The method of any one of embodiments 1 to 50, wherein the anti-BTLA antibody or antigen binding fragment thereof comprises a light chain variable region (LCVR) sequence of SEQ ID NO: 7 and a heavy chain variable region (HCVR) sequence of SEQ ID NO: 8.
53. The method of any one of embodiments 1 to 50, wherein the anti-BTLA antibody or antigen binding fragment thereof comprises a light chain sequence of SEQ ID NO: 9 and a heavy chain sequence of SEQ ID NO: 10.
54. The method of any one of embodiments 1 to 53, wherein the anti-BTLA antibody or antigen binding fragment thereof is in a composition comprising the effective amount of anti-BTLA antibody or antigen binding fragment thereof and a solubilizer and a stabilizer in a solution.
55. The method of embodiment 54, wherein the solubilizer comprising polysorbate 80.
56. The method of embodiment 54, wherein the stabilizer comprising the combination of sodium chloride and trehalose.
57. The method of any one of embodiments 1 to 53, wherein the anti-BTLA antibody or antigen binding fragment thereof is in a formulation comprising 10-30 mM histidine buffer, 30-100 mM sodium chloride, 50-200 mM trehalose, 0.01%-0.05% polysorbate 80, 10-50 mg/ml anti-BTLA antibody or antigen binding fragment thereof, wherein the histidine buffer comprises a molar ratio of 1:2, 1:3, 1:4 or 1:5 for histidine to histidine hydrochloride.
58. The method of embodiment 57, wherein the formulation comprises about 20 mM histidine buffer, about 50 mM sodium chloride, about 140 mM trehalose, about 0.02% polysorbate 80, about 20 mg/ml anti-BTLA antibody or antigen binding fragment thereof, wherein the histidine buffer comprises a molar ratio of 1:3 for histidine to histidine hydrochloride.
59. The method of any one of embodiments 1 to 58, wherein the anti-BTLA antibody or antigen binding fragment thereof exhibits a half life between about 7 and about 25 days.
60. The method of any one of embodiments 1 to 58, wherein the anti-BTLA antibody or antigen binding fragment thereof exhibits a half life about 6, 7, 8, 9, 10, 11, 12, 13, 14, 15, 16, 17, 18, 19, 20, 21, 22, 23, 24, 25, 26, 27, 28, 29 or 30 days.
61. The method of any one of embodiments 1 to 60, wherein lower than about 70%, 60%, 50%, 40%, 30%, 25%, 20% or 10% patients exhibit grade 3 or higher treatment emergent adverse event (TEAEs) from the treatment.
62. The method of any one of embodiments 1 to 60, wherein 10%-70%, 10%-60%, 10%-50%, 10%-40%, 10%-30%, 10%-25%, 10%-20% patients exhibit grade 3 or higher treatment emergent adverse event (TEAEs) from the treatment.
63. The method of any one of embodiments 1 to 61, wherein the method further comprising administering to the patient an effective amount of an anti-PD-1 or anti-PD-L1 antibody or antigen binding fragment thereof.
64. The method of embodiment 63, wherein the anti-PD-1 antibody is selected from the group consisting of nivolumab, pembrolizumab, toripalimab, sintilimab, camrelizumab, tislelizumab, and cemiplimab.
65. The method of embodiment 64, wherein the anti-PD-1 or anti-PD-L1 antibody is administered at a dose of about 240 mg.
66. The method of embodiment 64, wherein the anti-PD-1 or anti-PD-L1 antibody is administered at a dose of about 240 mg, Q3W.
67. The method of any one of embodiments 63 to 65, wherein the (i) anti-BTLA antibody or antigen binding fragment thereof and (ii) anti-PD-1 or anti-PD-L1 antibody or antigen binding fragment thereof are sequentially administered to the patient with the administration are spaced apart no more than 4, 6, 8, 10, 12 or 24 hours, or less than 1, 2, 3, 4, 5 or 6 days.
68. The method of any one of embodiments 1 to 65, wherein the (i) anti-BTLA antibody or antigen binding fragment thereof and (ii) anti-PD-1 or anti-PD-L1 antibody or antigen binding fragment thereof are co-formulated.
69. A method of suppressing tumor growth in a patient in need thereof comprising administering to the patient an effective amount of an anti-BTLA antibody or antigen binding fragment thereof in combination with an effective amount of an anti-PD-1 or anti-PD-L1 antibody or antigen binding fragment thereof, wherein the anti-BTLA antibody or antigen binding fragment thereof and the anti-PD-1 or anti-PD-L1 antibody suppress the tumor growth in the patient.
70. The method of embodiment 69, wherein the anti-PD-1 antibody is selected from the group consisting of nivolumab, pembrolizumab, toripalimab, sintilimab, camrelizumab, tislelizumab, and cemiplimab.
71. The method of embodiment 69 or 70, wherein the patient is administered intravenously with an anti-BTLA antibody or antigen binding fragment thereof at a weight based dose of 0.3-10 mg/kg Q3W or at a flat dose of about 20-500 mg Q3W in combination with an anti-PD-1 or anti-PD-L1 antibody or antigen binding fragment thereof at a dose of about 240 mg.
72. The method of any one of embodiments 69 to 71, wherein the patient is administered intravenously with an anti-BTLA antibody or antigen binding fragment thereof at a weight based dose of about 0.3, 1, 3, or 10 mg/kg Q3W or at a flat dose of about 20, 70, 200 or 500 mg Q3W in combination with an anti-PD-1 or anti-PD-L1 antibody or antigen binding fragment thereof at a dose of about 240 mg.
73. The method of any one of embodiments 69 to 71, wherein the anti-BTLA antibody or antigen binding fragment thereof is administered intravenously at a weight-based dose of about 3 mg/kg Q3W or at a flat dose of about 200 mg Q3W in combination with an anti-PD-1 or anti-PD-L1 antibody or antigen binding fragment thereof at a doses of about 240 mg.
74. A method for treating a melanoma in a patient in need thereof, comprising administering to the patient a pharmaceutically effective amount of an anti-BTLA antibody or antigen binding fragment thereof.
75. A method for treating a colorectal cancer (CRC) in a patient in need thereof, comprising administering to the patient a pharmaceutically effective amount of an anti-BTLA antibody or antigen binding fragment thereof.
76. A method for treating sarcoma in a patient in need thereof, comprising administering to the patient a pharmaceutically effective amount of an anti-BTLA antibody or antigen binding fragment thereof.
77. A method for treating a neuroendocrine tumor (NET) in a patient in need thereof, comprising administering to the patient a pharmaceutically effective amount of an anti-BTLA antibody or antigen binding fragment thereof.
78. A method for treating a squamous cell carcinoma (SCC) of the parotid gland in a patient in need thereof, comprising administering to the patient a pharmaceutically effective amount of an anti-BTLA antibody or antigen binding fragment thereof.
79. A method for treating a lymphoma in a patient in need thereof, comprising administering to the patient a pharmaceutically effective amount of an anti-BTLA antibody or antigen binding fragment thereof.
80. A method for treating a follicular lymphoma in a patient in need thereof, comprising administering to the patient a pharmaceutically effective amount of an anti-BTLA antibody or antigen binding fragment thereof.
81. A method for treating a Hodgkin's lymphoma in a patient in need thereof, comprising administering to the patient a pharmaceutically effective amount of an anti-BTLA antibody or antigen binding fragment thereof.
82. A method for treating a diffuse large B cell lymphoma (DLBCL) in a patient in need thereof, comprising administering to the patient a pharmaceutically effective amount of an anti-B TLA antibody or antigen binding fragment thereof.
83. A method for treating a solid tumor in a patient in need thereof, the method comprising: administering to the patient a pharmaceutically effective amount of an inhibitor of the interaction between the BTLA receptor and one or more of its ligands.
84. The method of embodiment 83, wherein the inhibitor inhibits the interaction between the BTLA receptor and herpesvirus entry mediator (HVEM).
85. The method of embodiment 83 or 84, wherein the method further comprising administering to the patient a pharmaceutically effective amount of an inhibitor of the interaction between the PD-1 receptor and one or more of its ligands.
86. The method of embodiment 85, wherein the PD-1 ligand is PD-L1 or PD-L2.
87. The method of any one of embodiments 83 to 86, wherein the solid tumor is selected from the group consisting of melanoma, colorectal cancer (CRC), sarcoma, neuroendocrine tumor (NET), squamous cell carcinoma (SCC) of the parotid gland, head and neck squamous cell carcinoma (HNSCC), follicular lymphoma, Hodgkin's lymphoma, diffuse large B cell lymphoma (DLBCL).
88. A method of suppressing tumor growth in a patient in need thereof comprising administering to the patient an effective amount of an anti-BTLA antibody or antigen binding fragment thereof in combination with an effective amount of a chemotherapeutic drug, wherein the anti-BTLA antibody and the chemotherapeutic drug suppress the tumor growth in the patient and wherein the anti-BTLA antibody or antigen binding fragment thereof inhibits and interaction between BTLA and HVEM in suppressing the tumor growth.
89. A method of suppressing metastasis tumor cells in a patient in need thereof comprising administering to the patient an effective amount of an anti-BTLA antibody or antigen binding fragment thereof.
90. A method of treating a HVEM-expressing solid tumor in a patient in need thereof, comprising administering to the patient a pharmaceutically effective amount of an anti-BTLA antibody or antigen binding fragment thereof, in combination with a pharmaceutically effective amount of one or more (i) chemotherapy drugs and (ii) an anti-PD-1 or anti-PD-L1 antibody or antigen binding fragment thereof.
91. A method of treating a tumor in a subject that is poorly responsive to or non-responsive to monotherapy with an anti-PD-1 or anti-PD-L1 antibody or an antigen-binding fragment thereof, the method comprising: administering an anti-BTLA antibody or antigen-binding fragment to the subject; and administering an anti-PD-1 or anti-PD-L1 antibody or antigen-binding fragment to the subject.

It will be readily apparent to those skilled in the art that other suitable modifications and adaptations of the methods described herein may be made using suitable equivalents without departing from the scope of the aspects disclosed herein. Having now described certain aspects in detail, the same will be more clearly understood by reference to the following example, which is included for purposes of illustration only and are not intended to be limiting. All patents, patent applications and references described herein are incorporated by reference in their entireties for all purposes.

### EXAMPLES

Example 1: Phase 1 study to evaluate TAB004 (icatolimab, also known as tifcemalimab)

A Phase 1, open-label, four-part study is conducted to evaluate the safety, tolerability, pharmacokinetics, immunogenicity, and antitumor activity of TAB004 as monotherapy and in combination with toripalimab in adult patients with advanced solid malignancies, including lymphoma. One cycle is 21 days (3 weeks) which includes TAB004 being administered via intravenous (IV) injection once every three weeks (Q3W). All subjects are treated until disease progression per RECIST v1.1 and iRECIST, or RECIL 2017 for lymphoma, or intolerable toxicity per CTCAE 5.0, withdrawal of consent, or end of the study, whichever occurs first. Disease progression must be confirmed at least 4 weeks but no longer than 8 weeks after initial documentation of progression.

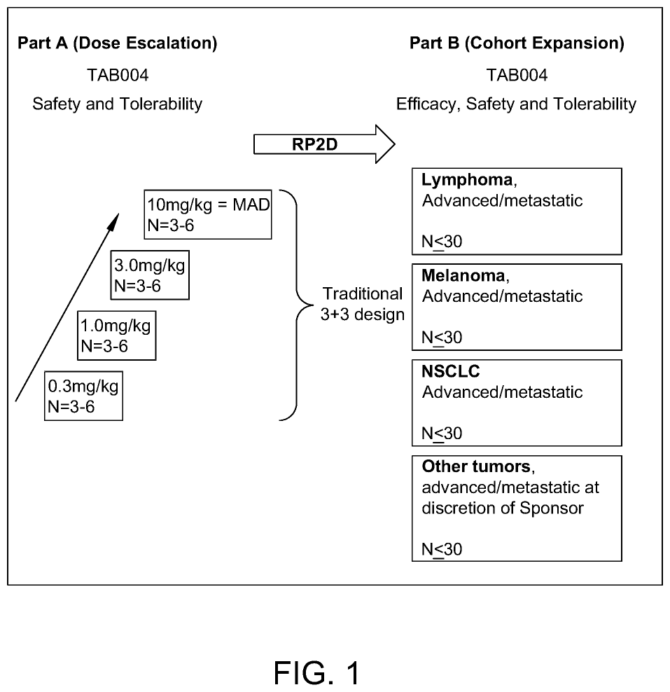

The four-part study consists of Parts A and C (dose-escalation), and Parts B and D (cohort expansion). Parts A and B evaluate TAB004 monotherapy administered intravenously, Q3W (FIG. 1); Parts C and D evaluate TAB004 when administered in combination with 240 mg toripalimab intravenously, Q3W.

Approximately 12-24 subjects are enrolled in Part A of this study, and up to 120 subjects are enrolled in Part B of this study. Additional subjects are recruited based on emerging safety and efficacy data from this trial. Subjects must meet all the following inclusion criteria to be eligible for participation in this study:
1. Subject is able to understand and willing to sign the Informed Consent Form (ICF).
2. Subject is male or female≥18 years.
3. Subject is confirmed histologically or cytologically to have advanced unresectable or metastatic solid tumor, including lymphoma. In Part A, subjects must have received, or be ineligible for or intolerant of all available approved or standard therapies known to confer clinical benefit including immunotherapy, or for whom no standard therapy exists. In Part B, subjects with advanced solid tumors, including but not limited to lymphoma, melanoma, NSCLC, or other tumors with agreement of the Sponsor, who must have received at least one line of therapy for advanced or metastatic disease, but are not required to have received all standard therapies known to confer clinical benefit.
4. Subject has measurable disease per RECISTv1.1 and iRECIST, or RECIL 2017 for lymphoma.
5. Subject has an ECOG performance status of 0 or 1 with life expectancy of ≥3 months in the opinion of the investigator.
6. Subject has adequate organ and marrow function, as defined below:
a) Hemoglobin≥8.0 g/dL within first 2 weeks prior to first dose of TAB004 (transfusion allowed)
b) Absolute neutrophil count (ANC)³ 1.0×109/L (1,000/mm³)
c) Platelet count≥75×109/L (75,000/mm³)
d) Total bilirubin≤1.5×ULN except subjects with documented Gilbert's syndrome who must have a baseline conjugated bilirubin≤3.0 mg/dL
e) Alanine aminotransferase (ALT) and aspartate aminotransferase (AST)≤2.5×ULN; for subjects with hepatic metastases, ALT and AST≤5×ULN
f) Serum creatinine≤1.5×ULN OR calculated creatinine clearance (CrCl) or 24-hour urine CrCl≥40 mL/minute
g) Cockcroft-Gault formula will be used to calculate CrCl. 24-hour urine CrCl will be derived using the measured creatinine clearance formula
h) International normalized ratio (INR)≤2.0 and activated partial thromboplastin time (aPTT)≤1.5×ULN; applies only to subjects who do not receive therapeutic anticoagulation; subjects receiving therapeutic anticoagulation (such as low molecular weight heparin or warfarin) should be on a stable dose
7. Subject is willing to provide consent for biopsy samples. In Part A, fresh pre-treatment biopsies will be requested from subjects with safely accessible lesions. For subjects who cannot provide a fresh pre-treatment biopsy, an archival specimen will be required. In Part B, fresh pre-treatment biopsies will be required from subjects with safely accessible lesions. Archival specimens will be requested.
8. Subject who is a female of childbearing potential who is sexually active with a non-sterilized male partner must use effective contraception from time of screening, and must agree to continue using such precautions for 90 days after the final dose of TAB004; cessation of birth control after this point should be discussed with a responsible physician. Periodic abstinence, the rhythm method, and the withdrawal method are not acceptable methods of birth control.
9. Subject who is a females of childbearing potential is defined as one who is not surgically sterile (i.e., bilateral tubal ligation, bilateral oophorectomy, or complete hysterectomy) or postmenopausal (defined as at least 12 months with no menses confirmed by follicle stimulating hormone [FSH] levels. FSH testing will be conducted at the Screening visit to confirm post-menopausal status).
10. Subject must use effective contraception.
11. Subject who is a non-sterilized male who is sexually active with a female partner of childbearing potential must use effective contraception from Day 1 and for 90 days after receipt of the final dose of TAB004.

Part A: dose-escalation; TAB004 monotherapy administered intravenously: In part A of this phase I study (NCT04137900), a total of 25 patients with solid tumor are enrolled. The median age is 62 (range 32-85) years with 16 (64%) male patients (FIG. 1). Patients are heavily pretreated with a median of 4 prior lines of therapy. Fifteen (60%) patients have received and progressed upon prior anti-PD-1/L1 therapy. Dose escalation (Part A) is conducted using a traditional 3+3 design. This is administered intravenously at doses of 0.3, 1, 3, and 10 mg/kg, Q3W.

Part B: cohort expansion; TAB004 monotherapy administered intravenously: In Part B, 3 mg/kg and 200 mg flat dose are selectively guided by pharmacokinetics, pharmacodynamics, safety, and preliminary efficacy data (FIG. 1). In Part B, up to 50 patients are enrolled in each advanced solid tumor indication (up to 200 patients in total) that may include but not be limited to lymphoma, melanoma, NSCLC, or other advanced tumors with agreement of the Sponsor. The first 18 patients enrolled in Part B receive the Recommended Phase II dose (RP2D) of 3 mg/kg. Thereafter, patients enrolled into Part B receive a 200 mg flat dose of TAB004 equivalent to the weight based RP2D dose. Taking into account the drug packaging of 100 mg/6 mL/vial, and an average patient weight of 70 kg, the equivalent flat dose of 200 mg Q3W does not pose unreasonable risk to patients and is evaluated in parallel to the 3 mg/kg RP2D being considered in Part B.

Part C: dose-escalation; TAB004 when administered in combination with 240 mg toripalimab intravenously: In part C, Dose escalation is initiated in parallel to the ongoing recruitment of Part B. Part C is conducted using a traditional 3+3 design and recruit patients with advanced unresectable or metastatic solid tumors, including lymphoma, who must have received at least one line of therapy for advanced or metastatic disease. All patients receive the standard 240 mg dose of toripalimab and TAB004 will be administered intravenously at doses of 20, 70, 200 and 500 mg Q3W. Dose escalation continues until the MTD for the TAB004 and toripalimab combination regimen is reached. Alternatively, if an MTD is not reached, dose escalation is continued until the MAD of 500 mg TAB004 is reached. Part D: cohort expansion; TAB004 when administered in combination with 240 mg toripalimab intravenously

Part D: cohort expansion; TAB004 when administered in combination with 240 mg toripalimab intravenously: In Part D, up to 50 patients are enrolled in each advanced solid tumor indication (up to 250 patients in total) that may include but not be limited to lymphoma, melanoma, NSCLC, RCC or UC.

### Example 2: TAB004 Pharmacokinetics and Pharmacodynamics Study

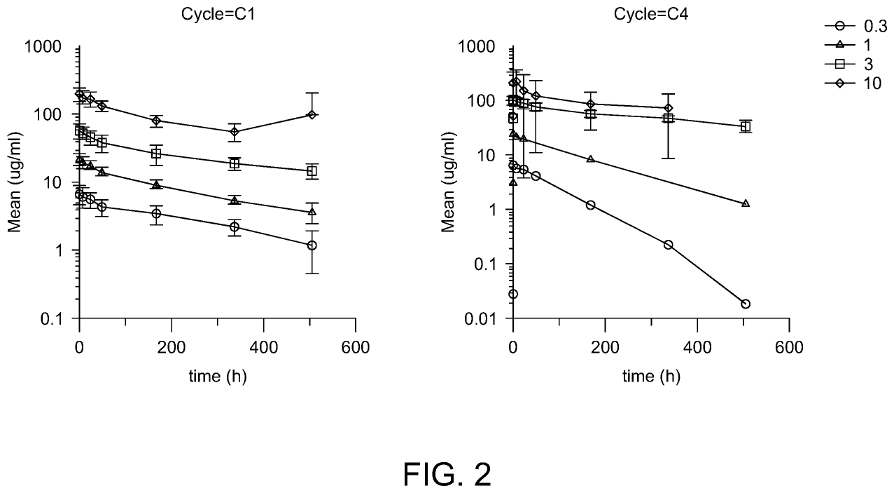

Mean serum TAB004 concentration time profiles following a first dose (Cycle 1) and after consecutive Q3W doses (Cycle 4) are presented in FIG. 2.

After single-dose intravenous infusion of TAB004 to the patients at different doses (0.3, 1, 3 and 10 mg/kg) respectively, the pharmacokinetic data shows that the pharmacokinetic (PK) profile of TAB004 exhibited linear pharmacokinetic characteristics in the dose range of 0.3-10 mg/kg, with the serum TAB004 exposure increasing at a dose-proportional manner. The peak concentration of TAB004 are 7.01±2.02 ug/mL, 23.24±3.55 ug/mL, 59.90±14.30 ug/mL and 210.49±35.89 ug/mL and the AUC(0-t) are 1529.85±381.57 hr*μg/mL, 4491.37±529.25 hr*μg/mL, 12752.43±3305.86 and 38683.42±8498.07 hr*μg/mL for 0.3, 1, 3 and 10 mg/kg cohorts respectively. The observed serum half-life of TAB004 is 251 hours (10.5 days) to 433 hours (18.0 days) after the first administration of doses of 0.3, 1, 3 or 10 mg/kg. The results of clearance (Cl) in various dose groups are similar, being 0.21 mL/hr/kg, 0.23 mL/hr/kg, 0.24 mL/hr/kg and 0.26 mL/hr/kg, respectively. The CL and Vd of TAB004 concur with the pharmacokinetic characteristics of IgG4 antibody in humans.

Steady-state concentration is achieved when TAB004 is administered at 4 consecutive doses Q3W, with the mean minimum observed concentration (Cmin) being 0.019 μg/mL(n=1), 1.29 μg/mL(n=1), 35.18 μg/mL(n=5) and 65.82 μg/mL(n=2) respectively. the Cmax is 6.77 μg/mL, 25.68 μg/mL, 94.09±23.36 and 308.42±30.65 μg/mL, respectively, with an AUC (0-t) of 735.46 hr*μg/mL, 4270.05 hr*μg/mL, 24110.38±7107.78 hr*μg/mL and 54983.93 hr*μg/mL, respectively. The accumulation factor is in the range from 1.0 to 2.0 in the dose groups, indicating that there is a slight accumulation of TAB004 in the body after multiple-dose administration.

A pooled PK analysis is performed by pooling the sample data of TAB004 in various dose groups (doses 0.3-10 mg/kg) in three phase I dose-escalation clinical studies (TAB004-01, JS004-001-I and J5004-002-I). The study results show that the peak concentration (Cmax) and area under the plasma concentration versus time curve (AUCO-t) of TAB004 increased dose proportionally in the dose range of 0.3 to 10 mg/kg every 3 weeks. TAB004 show an obvious “long-acting” characteristic of antibody drugs with long half-life. The serum drug concentrations basically reach a steady state after about 4 consecutive IV infusions of TAB004 to the patients, with half-life (t½) of 410.17±193.52 hours (17.1±8.1 days), Cmax of 94.15±25.50 μg/mL, Cmin of 28.70±12.80 μg/mL and AUC(0-t) of 41854.04±16891.05 h. μg/mL, respectively, after multiple administrations in the 3 mg/kg group (n=8).

In summary, Cmax and AUC increase at a dose-proportional manner. The mean elimination half-life of TAB004 is 7.5 to 19.2 days in four dose cohorts. Steady-state concentration is achieved if TAB004 is administered 4 times Q3W. Average trough concentrations (Ctrough) of TAB004 are 0.019, 1.29, 33.13 and 65.82 μg/ml at dose of 0.3, 1, 3 and 10 mg/kg respectively. At steady state, 3 mg/kg Q3W TAB004 trough concentration is 33.13 μg/ml, about 10-fold higher than saturation concentration.

BTLA receptor occupancy after intravenous infusion of TAB004 to the patients at different doses are characterized. Result show that full BTLA receptor occupancy (>80% RO) is observed in all dosage groups (0.3, 1, 3 and 10 mg/kg Q3W) and complete occupancy of BTLA receptors could be maintained during the whole course of treatment in all patients dosed Q3W with TAB004 above 1 mg/kg dose level.

### Example 3: TAB004 Safety Profiles

All AEs described below refer to treatment-emergent adverse events (TEAEs). The safety profile in this study is summarized by dose levels. A total of 31 patients (31/33, 93.9%) experienced at least 1 adverse event (AE) and 45.5% (15/33) of the patients experienced study drug-related AEs as assessed by the investigators (Table 3). Serious adverse events (SAEs) are reported by 7 patients (21.2%). Further, 5 patients experienced AEs leading to study drug interruption; 4 patients experienced AEs that led to treatment discontinuation. No infusion reaction is reported during the study.

TABLE 3
Summary of Adverse Events

| | TAB004 | | TAB004 | | TAB004 | | TAB004 | | TOTAL | |
|---|---|---|---|---|---|---|---|---|---|---|
| | 0.3 mg/kg | | 1 mg/kg | | 3 mg/kg | | 10 mg/kg | | TAB004 | |
| | (N = 3) | | (N = 3) | | (N = 18) | | (N = 9) | | (N = 33) | |
| Treatment emergent | 3 | (100.0) | 3 | (100.0) | 16 | (88.9) | 9 | (100.0) | 31 | (93.9) |
| adverse event (TEAE) | | | | | | | | | | |
| Related to study drug | 2 | (66.7) | 1 | (33.3) | 7 | (38.9) | 5 | (55.6) | 15 | (45.5) |
| Dose limiting toxicity | 0 | | 0 | | 0 | | 0 | | 0 | |
| (DLT) | | | | | | | | | | |
| Serious adverse event | 1 | (33.3) | 2 | (66.7) | 1 | (5.6) | 3 | (33.3) | 7 | (21.2) |

| Related to study drug | 0 | 0 | | 0 | | 1 | (11.1) | 1 | (3.0) |
|---|---|---|---|---|---|---|---|---|---|
| Immune related adverse | 0 | 1 | (33.3) | 3 | (16.7) | 1 | (11.1) | 5 | (15.2) |
| event (irAE) | | | | | | | | | |

| | | | | | | | | |
|---|---|---|---|---|---|---|---|---|
| Adverse events leading | 0 | 0 | 2 | (11.1) | 3 | (33.3) | 5 | (15.2) |
| to dose interruption | | | | | | | | |

| Related to study drug | 0 | | 0 | 0 | 2 | (22.2) | 2 | (6.1) |
|---|---|---|---|---|---|---|---|---|
| Adverse events leading | 1 | (33.3) | 0 | 0 | 3 | (33.3) | 4 | (12.1) |
| to dose discontinuation | | | | | | | | |

| | | | | | | | |
|---|---|---|---|---|---|---|---|
| Related to study drug | 0 | 0 | 0 | 1 | (11.1) | 1 | (3.0) |

The most frequently reported AEs (reported by ≥5% patients) are fatigue (30.3%), anaemia (18.2%), abdominal pain (15.2%), arthralgia (15.2%), constipation (12.1%), nausea (12.1%), pruritus (12.1%), back pain (9.1%), blood creatinine increased (9.1%), contusion (9.1%), diarrhoea (9.1%), myalgia (9.1%), abdominal pain upper (6.1%), alanine aminotransferase increased (6.1%), aspartate aminotransferase increased (6.1%), blood alkaline phosphatase increased (6.1%), bone pain (6.1%), chills (6.1%), cough (6.1%), decreased appetite (6.1%), hypoalbuminaemia (6.1%), hypomagnesaemia (6.1%), hyponatraemia (6.1%), insomnia (6.1%), lipase increased (6.1%), lymphedema (6.1%), pain (6.1%), paraesthesia (6.1%), rash maculo-papular (6.1%), tumor pain (6.1%), urinary tract infection (6.1%) and urticaria (6.1%).

The most frequently reported drug-related AEs (reported by >5% patients) are fatigue (18.2%), pruritus (12.1%), diarrhoea (9.1%), myalgia (9.1%), nausea (9.1%), bone pain (6.1%), pain (6.1%), tumour pain (6.1%) and urticaria (6.1%), see Table 4.

TABLE 4
Summary of Treatment-Related TEAE by SOC and PT

| | | | TAB004 | TAB004 | TAB004 | | TAB004 |
|---|---|---|---|---|---|---|---|
| MedDRA SOC | Overall | | 0.3 mg/kg | 1 mg/kg | 3 mg/kg | | 10 mg/kg |
| PT | (N = 33) | | (N = 3) | (N = 3) | (N = 18) | | (N = 9) |
| Number of Patients | 15 | (45.5) | 2 (66.7) | 1 (33.3) | 7 | (38.9) | 5 (55.6) |
| with at least one | | | | | | | |
| TEAE related to | | | | | | | |
| study drug | | | | | | | |
| Gastrointestinal disorders | 9 | (27.3) | 2 (66.7) | 0 | 2 | (11.1) | 5 (55.6) |
| Diarrhea | 3 | (9.1) | 1 (33.3) | 0 | 0 | | 2 (22.2) |
| Na [symbol] | 3 | (9.1) | 1 (33.3) | 0 | 0 | | 2 (22.2) |
| Abdominal pain | 1 | (3.0) | 0 | 0 | 0 | | 1 (11.1) |
| Abdominal pain upper | 1 | (3.0) | 0 | 0 | 1 | (5.6) | 0 |
| Dry mouth | 1 | (3.0) | 0 | 0 | 0 | | 1 (11.1) |
| D [symbol] | 1 | (3.0) | 0 | 0 | 1 | (5.6) | 0 |
| Dysphagia | 1 | (3.0) | 0 | 0 | 0 | | 1 (11.1) |
| Enterocolitis | 1 | (3.0) | 0 | 0 | 0 | | 1 (11.1) |
| Gastroesophageal | 1 | (3.0) | 0 | 0 | 0 | | 1 (11.1) |
| reflux disease | | | | | | | |
| Pancreatitis | 1 | (3.0) | 0 | 0 | 0 | | 1 (11.1) |
| General disorders | 8 | (24.2) | 1 (33.3) | 1 (33.3) | 3 | (16.7) | 3 (33.3) |
| and administration | | | | | | | |
| site conditions | | | | | | | |
| Fatigue | 6 | (18.2) | 1 (33.3) | 1 (33.3) | 3 | (16.7) | 1 (11.1) |
| Pain | 2 | (6.1) | 0 | 0 | 1 | (5.6) | 1 (11.1) |
| Chills | 1 | (3.0) | 0 | 0 | 0 | | 1 (11.1) |
| Cyst | 1 | (3.0) | 0 | 0 | 0 | | 1 (11.1) |
| Pyrexia | 1 | (3.0) | 0 | 1 (33.3) | 0 | | 0 |
| Skin and subcutaneous | 7 | ( [symbol] ) | 1 (33.3) | 1 (33.3) | 4 | (22.2) | 1 (11.1) |
| tissue disorders | | | | | | | |
| P [symbol] | 4 | (12.1) | 1 (33.3) | 0 | 2 | (11.1) | 1 (11.1) |
| U [symbol] | 2 | (6.1) | 0 | 1 (33.3) | 1 | (5.6) | 0 |
| Rash [symbol] | 1 | (3.0) | 0 | 0 | 1 | (5.6) | 0 |
| Skin [symbol] | 1 | (3.0) | 0 | 0 | 1 | (5.6) | 0 |
| Musculoskeletal and | 6 | (18.2) | 2 (66.7) | 0 | 2 | (11.1) | 2 (22.2) |
| convective tissue disorders | | | | | | | |
| M [symbol] | 3 | (9.1) | 1 (33.3) | 0 | 0 | | 2 (22.2) |
| Bone pain | 2 | (6.1) | 1 (33.3) | 0 | 1 | (5.6) | 0 |
| A [symbol] | 1 | (3.0) | 0 | 0 | 1 | (5.6) | 0 |
| Arthritis | 1 | (3.0) | 1 (33.3) | 0 | 0 | | 0 |
| Back pain | 1 | (3.0) | 1 (33.3) | 0 | 0 | | 0 |
| Inves [symbol] | 3 | (9.1) | 0 | 0 | 1 | (5.6) | 2 (22.2) |
| Blood thyroid | 1 | (3.0) | 0 | 0 | 1 | (5.6) | 0 |
| stimulating hormone | | | | | | | |
| increased | | | | | | | |
| Lipase increased | 1 | (3.0) | 0 | 0 | 0 | | 1 (11.1) |
| Weight decreased | 1 | (3.0) | 0 | 0 | 0 | | 1 (11.1) |
| Respiratory, thoracic | 3 | (9.1) | 0 | 1 (33.3) | 1 | (5.6) | 1 (11.1) |
| and med [symbol] disorders | | | | | | | |
| Cough | 1 | (3.0) | 0 | 1 (33.3) | 0 | | 0 |
| Dy [symbol] | 1 | (3.0) | 0 | 0 | 1 | (5.6) | 0 |
| Rhinorr [symbol] | 1 | (3.0) | 0 | 0 | 0 | | 1 (11.1) |
| Blood and lymphatic | 2 | (6.1) | 0 | 0 | 1 | (5.6) | 1 (11.1) |
| system disorders | | | | | | | |
| A [symbol] | 1 | (3.0) | 0 | 0 | 1 | (5.6) | 0 |
| Lymphopenia | 1 | (3.0) | 0 | 0 | 0 | | 1 (11.1) |
| Injury, p [symbol] and | 2 | (6.1) | 0 | 0 | 1 | (5.6) | 1 (11.1) |
| procedural complications | | | | | | | |
| Con [symbol] usion | 1 | (3.0) | 0 | 0 | 0 | | 1 (11.1) |
| Epicondylitis | 1 | (3.0) | 0 | 0 | 1 | (5.6) | 0 |
| Neoplasms benign, malignant | 2 | (6.1) | 1 (33.3) | 0 | 1 | (5.6) | 0 |
| and unspecified (incl cysts | | | | | | | |
| and polyp [symbol] ) | | | | | | | |
| Tumour pain | 2 | (6.1) | 1 (33.3) | 0 | 1 | (5.6) | 0 |
| tumour [symbol] | 1 | (3.0) | 0 | 0 | 1 | (5.6) | 0 |
| Nervous system | 2 | (6.1) | 1 (33.3) | 1 (33.3) | 0 | | 0 |
| disorders | | | | | | | |
| Burning sensation | 1 | (3.0) | 1 (33.3) | 0 | 0 | | 0 |
| P [symbol] | 1 | (3.0) | 0 | 1 (33.3) | 0 | | 0 |
| Eye disorders | 1 | (3.0) | 0 | 0 | 1 | (5.6) | 0 |
| Photophobia | 1 | (3.0) | 0 | 0 | 1 | (5.6) | 0 |

| | | | | | | |
|---|---|---|---|---|---|---|
| Hep [symbol] disorders | 1 | (3.0) | 0 | 0 | 0 | 1 (11.1) |
| Bile duct obstruction | 1 | (3.0) | 0 | 0 | 0 | 1 (11.1) |
| Vascular disorders | 1 | (3.0) | 1 (33.3) | 0 | 0 | 0 |
| Fl [symbol] shing | 1 | (3.0) | 1 (33.3) | 0 | 0 | 0 |

[symbol]

indicates data missing or illegible when filed

Grade 3 or above AEs are experienced by 9 patients (27.3%), and 4 of them are in the 10 mg/kg cohort. The reported grade 3 or above AEs are abdominal pain, constipation, intestinal obstruction, small intestinal obstruction, back pain, fatigue, blood alkaline phosphatase increased, lipase increased, lymphocyte count decreased, hyponatraemia, hypercalcaemia, haemoptysis, anaemia, lymphopenia, urinary tract infection, escherichia sepsis and bile duct obstruction each.

Serious adverse events (SAEs) are experienced by 7 patients (21.2%), and 3 of them are in the 10 mg/kg, 2 in 1 mg/kg and 1 in 0.3 mg/kg and 3 mg/kg each. All SAEs are assessed by investigators as unlikely related to or not related to study drug.

By the data cutoff date of December 31, 2021, the median follow-up is 32 weeks. No DLT was observed. 24 (96%) patients experienced treatment emergent adverse event (TEAEs), with 7 (28%) experienced grade 3 TEAEs. No grade 4 or 5 TEAE occurred. The incidence or severity of AE is not associated with the dose. The most common TEAEs are fatigue (32%), abdominal pain (20%), diarrhea (16%), arthralgia (16%), aspartate aminotransferase increased (16%), constipation (16%), and contusion (16%). One (4%) TEAE leads to discontinuation of study drug. Four (16%) patients experienced immune related AE.

### Example 4: TAB004 Exhibits Efficacy in Treating Melanoma and Various Other Indications

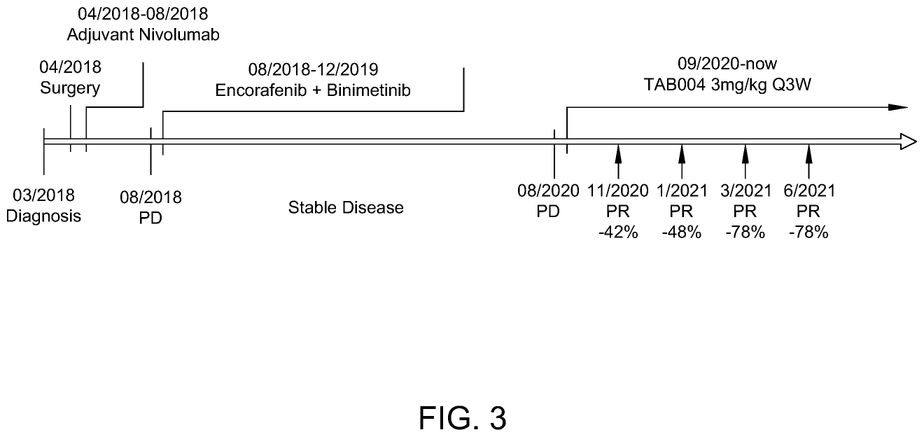

Among 19 evaluable patients by the cutoff date, one confirmed partial response (PR) (a melanoma patient in the 3 mg/kg dosing group) and 6 stable disease (SD) (two colorectal cancer (CRC) patients with one in each of the 0.3 mg/kg and 3 mg/kg dosing groups, one sarcoma patient in the 3 mg/kg dosing group, one neuroendocrine tumor (NET) patient in the 10 mg/kg dosing group, one patient with squamous cell carcinoma (SCC) of the parotid gland in the 1 mg/kg dosing group, one head and neck squamous cell carcinoma (HNSCC) patient in the 10 mg/kg dosing group) are observed as assessed by the investigator per RECIST v1.1. BTLA receptor is fully occupied in the 3 and 10 mg/kg cohorts. By the data updated on Jan. 19, 2022, there is one confirmed PR in a melanoma patient (with tumor shrinkage of −42% and −48% for the first two evaluation points at 2 and 4 months after the first TAB004 dose; followed by −78% in the next two evaluation points of 6 and 9 months after the first TAB004 dose) who is refractory to Nivolumab and Braf/Mek inhibitors (FIG. 3). The tumor of this patient has high expression of HVEM, PD-L1 and CD8 lymphocytes with IHC H-score 115, 50, and 90, respectively. In addition, tumor shrinkage is observed in 1 NET (−2%), 1 HNSCC (−12%) and 1 melanoma (−48%) patient.

### Example 5: Recommended Phase II Dose (RP2D) and Biomarker Analysis

By the data cutoff on Jan. 19, 2022, full occupancy (RO 80% or higher) is maintained for 3 mg/kg and 10 mg/kg Q3W cohorts. Based on PK, RO, and preliminary efficacy results, TAB004 3 mg/kg Q3W is the recommended Phase II dose. A flat dose of 200 mg Q3W is also being considered.

Biomarker analysis indicated co-expression of HVEM and CD8 is associated with favorable response. For example, among 9 patients with CD8 H score≥50, 1 patient achieves PR and 3 patients exhibit SD.

In all, TAB004 monotherapy is well tolerated in all doses evaluated and shows clinical efficacy as a monotherapy. TAB004 in combination with toripalimab (anti-PD-1) for the treatment of patients with advanced solid tumors is currently ongoing.

### Example 6 Phase 1 Study to Evaluate TAB004 (Tifcemalimab, Also Known as Icatolimab)

Another multi-center, open-label, 3-part (dose-escalation, cohort-expansion, and indication-expansion), phase I clinical study of TAB004/JS004 alone and in combination with toripalimab (JS001) is conducted in China to explore the safety, tolerability, pharmacokinetics, and efficacy of patients with relapsed/refractory (R/R) malignant lymphoma (NCT04477772).

In the monotherapy regiment, TAB004 is administered using a 3+3 design with escalating doses of 1, 3 and 10 mg/kg intravenously Q3W and followed by 3 mg/kg and 200 mg dose expansion until disease progression or intolerable toxicity. In the combination regiment, patients receive ascending doses of TAB004 (100 mg and 200 mg) plus toripalimab (240 mg). Dose-limiting toxicity (DLT) is evaluated by a safety monitoring committee. Study objectives include safety, pharmacokinetics, and efficacy.

By the cutoff date of Oct. 26, 2022, a total of 63 patients are enrolled, including 25 in the monotherapy and 38 in the combination subgroup. The lymphoma subtypes include 43 Hodgkin's lymphoma (HL) and 20 non-Hodgkin's lymphoma. The median age is 39 (range 19-70) years with 45 (71.4%) male patients. 46 (73.0%) patients have been pretreated with a median of line 4 anti-PD-1/L1 prior therapy.

By the cutoff date of Oct. 26, 2022, the median follow-up time is 29.1 weeks. No DLT is observed in either monotherapy or combination dose escalation. Fifty-four (84.1%) patients experience treatment emergent adverse events (TEAEs), with 12 (19.0%) experience grade 3 or above TEAEs. The most common TEAEs are fever (22.2%) and anemia (20.6%) Two treatment-related adverse events (TRAEs) lead to treatment discontinuation. Ten (40.0%) and 20 (52.6%) patients experience immune related adverse events (AEs) in the monotherapy and combination subgroups, respectively, and 3 (4.8%) patients experienced≥grade 3 irAEs. The irAE profile of the combination was consistent with toripalimab monotherapy. No novel safety signals were identified in the combination cohorts.

Among 25 evaluable patients receiving monotherapy, 1 PR (follicular lymphoma in the 3 mg/kg dose group) and 7 SD (2 Hodgkin's lymphoma with one in each of the 1 mg/kg and 3mg/kg dose groups, 2 diffuse large B cell lymphoma (DLBCL) with one in each of the 3 mg/kg and 10 mg/kg dose groups, and 2 Hodgkin's lymphoma and 1 follicular lymphoma with all three being in the same 200 mg flat dose group) are observed as assessed by Lugano Criteria.

Among 28 evaluable patients receiving combination therapy, 24 (85.7%) patients progressed upon prior anti-PD-1 therapy. 1 CR, 10 PR having an ORR of 39.3% and 13 SD, having a DCR of 5.7% are observed.

Full BTLA receptor occupancy are observed in all evaluated doses. The mean half-life of TAB004 is 13.3 days. Biomarker analysis indicates that positive HVEM expression is associated with favorable response. In all, TAB004 alone or in combination with toripalimab are well tolerated in all doses evaluated and showed clinical efficacy in patients with R/R lymphoma.

### Example 7 Inhibition of Tumor Growth in Mice By the Combination of Anti-BTLA Antibody and Anti-PD-1 Antibody

The study relates to the establishment of MC38 colon cancer animal models of B-hPD-1/hBTLA humanized mice and synergistic anti-tumor effect of the anti-BTLA antibody and the anti-PD-1 antibody administered in combination. The B-hPD-1/hBTLA humanized mice are each subcutaneously inoculated, in the right dorsal side, with 0.1 mL of MC38 cells resuspended in PBS at a concentration of 1.0×106 cells/0.1 mL. The appropriate mice are selected according to the tumor volume and body weight of the mice and evenly distributed into 4 experimental groups, 5 to 6 mice in each group, and administration via intraperitoneal injection (IP) is started on the day of grouping. The specific administration regimen is shown in Table 5 below.

TABLE 5
Administration regimen.

| | Administered | | dosage | Route of | Frequency of | Number of |
|---|---|---|---|---|---|---|
| Group | antibody | Number of mice | (mg/kg)^{a} | administration | administration^{b} | administration |
| G1 | Saline | 6 | — | IP | BIW | 6 |
| G2 | JS001 | 5 | 3.75 | IP | BIW | 6 |
| G3 | JS004 | 5 | 3.75 | IP | BIW | 6 |
| G4^{c} | JS001 + JS004 | 6 | 3.75 + 3.75 | IP | BIW | 6 |

Note:
^{a}the administration volume is calculated at 10 μL/g according to the body weight of the experimental animal;
^{b}BIW means administering twice a week.

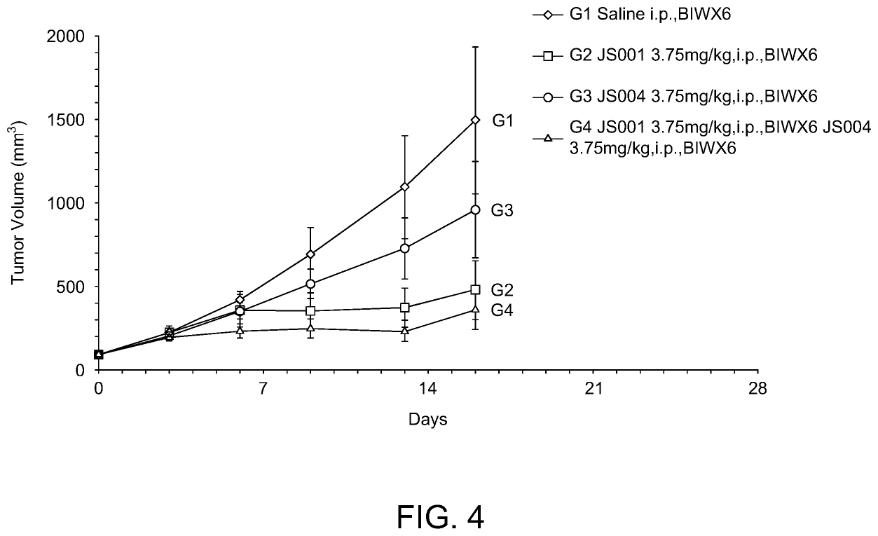

At the end of the 16-day after the first dosing, tumor weight and volume of each group of mice are measured, and relative tumor growth inhibition rates (TGI (%)) are calculated. Results are shown in Table 6 and FIG. 4. The results show that both the single and combination regimens are effective in inhibition of tumor growth especially for the combination regimen.

TABLE 6
Effect of JS004 and combination of JS001 and JS004 on tumor
tissue growth in tumor-bearing mice (mm3, mean ± SD)

| | | Mean | | |
|---|---|---|---|---|
| | | tumor | | |
| | dosage | volume | | |
| Group | (mg/kg)^{a} | (mm³) | TGI(%) | P value |
| G1 | — | 1496 | | |
| G2 | 3.75 | 475 | 72.6% | 0.078 |
| G3 | 3.75 | 959 | 38.2% | 0.357 |
| G4^{c} | 3.75 + 3.75 | 358 | 81.0% | 0.032 |

1. A method for treating a solid tumor in a patient in need thereof, the method comprising:
administering to the patient a pharmaceutically effective amount of an anti-BTLA antibody or antigen binding fragment thereof.

2. The method of claim 1, wherein the solid tumor is one or more tumors selected from the group consisting of melanoma, colorectal cancer (CRC), sarcoma, neuroendocrine tumor (NET), squamous cell carcinoma (SCC) of the parotid gland, head and neck squamous cell carcinomas (HNSCCs), follicular lymphoma, Hodgkin's lymphoma, and diffuse large B cell lymphoma (DLBCL).

3. The method of claim 1, wherein the solid tumor expresses herpes virus entry mediator (HVEM), and wherein at least about 1%, 5%, 10%, 15%, 20%, 30%, 40%, 50%, 60%, 70%, 80%, 90%, or 95% of tumor cells are positive for HVEM.

4. The method of claim 1, wherein the solid tumor co-expresses HVEM and CD8, and wherein at least about 1%, 5%, 10%, 15%, 20%, 30%, 40%, 50%, 60%, 70%, 80%, 90%, or 95% of tumor cells are positive for HVEM and CD8.

5. The method of claim 1, wherein the treatment with the anti-BTLA antibody or antigen binding fragment thereof is used as an adjuvant therapy.

6. The method of claim 1, wherein the patient receives at least one, two, three or four prior lines of therapy.

7. The method of claim 1, wherein the pharmaceutically effective amount of the anti-BTLA antibody or antigen binding fragment thereof is administered to the patient in combination with a pharmaceutically effective amount of one or more chemotherapy drugs.

8. The method of claim 1, wherein the anti-BTLA antibody or antigen binding fragment thereof is administered as an intravenous infusion.

9. The method of claim 1, wherein the anti-BTLA antibody or antigen binding fragment thereof comprising one or more properties selected from the following: A) completely blocking the binding of BTLA to HVEM; B) cross-reacting with cynomolgus monkey BTLA; C) binding to human BTLA with a K_{D}≤0.28 nM; and D) having no ability to mediate Antibody-Dependent Cellular Cytotoxicity (ADCC) effect.

10. The method of claim 1, wherein the anti-BTLA antibody or antigen binding fragment thereof comprises a light chain variable region of LCDR1 having the amino acid sequence of SEQ ID NO: 1, LCDR2 having the amino acid sequence of SEQ ID NO: 2, LCDR3 having the amino acid sequence of SEQ ID NO: 3; and further comprises a heavy chain variable region of HCDR1 having the amino acid sequence of SEQ ID NO: 4, HCDR2 having the amino acid sequence of SEQ ID NO: 5, HCDR3 having the amino acid sequence of SEQ ID NO:6.

11. The method of claim 1, wherein the anti-BTLA antibody or antigen binding fragment thereof comprises a light chain variable region sequence of SEQ ID NO: 7 and a heavy chain variable region sequence of SEQ ID NO: 8.

12. The method of claim 1, wherein the anti-BTLA antibody or antigen binding fragment thereof comprises a light chain sequence of SEQ ID NO: 9 and a heavy chain sequence of SEQ ID NO: 10.

13. The method of claim 1, wherein the anti-BTLA antibody or antigen binding fragment thereof is in a composition comprising the effective amount of anti-BTLA antibody or antigen binding fragment thereof and a solubilizer and a stabilizer in a solution.

14. The method of claim 1, wherein the method further comprising administering to the patient an effective amount of an anti-PD-1 or anti-PD-L1 antibody or antigen binding fragment thereof.

15. A method of suppressing tumor growth in a patient in need thereof comprising administering to the patient an effective amount of an anti-BTLA antibody or antigen binding fragment thereof in combination with an effective amount of an anti-PD-1 or anti-PD-L1 antibody or antigen binding fragment thereof, wherein the anti-BTLA antibody or antigen binding fragment thereof and the anti-PD-1 or anti-PD-L1 antibody or antigen binding fragment thereof suppress the tumor growth in the patient.

16. A method for treating a solid tumor in a patient in need thereof, the method comprising: administering to the patient a pharmaceutically effective amount of an inhibitor of the interaction between the BTLA receptor and one or more of its ligands.

17. The method of claim 16, wherein the inhibitor inhibits the interaction between the BTLA receptor and HVEM.

18. The method of claim 16, wherein the method further comprising administering to the patient a pharmaceutically effective amount of an inhibitor of the interaction between the PD-1 receptor and one or more of its ligands.

19. The method of claim 18, wherein the PD-1 ligand is PD-L1 or PD-L2.

20. The method of claim 16, wherein the solid tumor is selected from the group consisting of melanoma, CRC, sarcoma, NET, SCC of the parotid gland, HNSCC, follicular lymphoma, Hodgkin's lymphoma, and DLBCL.
